(19) United States
(12) Patent Application Publication
(10) Pub. No.: US 2024/0025969 A1
(43) Pub. Date: Jan. 25, 2024

(54) NPC1 MONOBODIES AND MONOBODY CONJUGATES THEREOF

(71) Applicant: NEW YORK UNIVERSITY, New York, NY (US)

(72) Inventors: Craig RAMIREZ, Austin, TX (US); Andrew HAUSER, New York, NY (US); Dafna BAR-SAGI, New York, NY (US); Akiko KOIDE, New York, NY (US); Shohei KOIDE, New York, NY (US)

(21) Appl. No.: 18/252,285

(22) PCT Filed: Nov. 10, 2021
(86) PCT No.: PCT/US2021/058783

§ 371 Date: May 9, 2023

Related U.S. Application Data
(60) Provisional application No. 63/112,031, filed on Nov. 10, 2020.

(51) Int. Cl. C07K 14/78; C07K 16/18; A61K 47/64

(52) U.S. Cl. CPC C07K 14/78; C07K 16/18; A61K 47/6435; C07K 2317/77; C07K 2318/20; C07K 2317/92; C07K 2317/73

(57) ABSTRACT

The present invention is directed to Niemann-Pick disease, type C1 (NPC1) binding polypeptides and NPC1 binding peptide conjugates comprising these binding polypeptides. The present invention is further directed to pharmaceutical compositions comprising these NPC1 binding polypeptide and binding peptide conjugates and the use of these compositions to treat a variety of conditions, including cancer, infectious diseases, neurodegenerative diseases, inflammatory conditions, and bone conditions. The NPC1 binding conjugates are also useful for enhancing endosomal release of pharmaceutically active moieties.

This application claims the priority benefit of U.S. Provisional Patent Application Ser. No. 63/112,031, filed Nov. 10, 2020, which is hereby incorporated by reference in its entirety.

### FIELD

The present invention is directed to Niemann-Pick disease, type C1 (NPC1) binding polypeptides and NPC1 binding peptide conjugates comprising these binding polypeptides. The present invention is further directed to pharmaceutical compositions comprising these NPC1 binding polypeptides and binding peptide conjugates and the use of these compositions in treating a variety of conditions.

### BACKGROUND

Niemann-Pick disease, type C1 (NPC1) is located on the membrane of endosomal compartments and is essential for cholesterol trafficking from the endosome to the plasma membrane. Interrupting cholesterol transport to the plasma membrane disrupts plasma membrane integrity, prevents proper Rac1 localization that is necessary for migration and metastasis, and potentially disrupts other cholesterol-dependent proteins such as receptor tyrosine kinases (RTKs). An additional observation is that NPC1 disruption in certain cancer cells blocks autophagic flux. This is clinically interesting because autophagy is a mechanism of treatment resistance to chemotherapy in cancers such as colorectal and pancreatic cancer.

Autophagy is a cellular process that assists advanced cancers with proliferation and survival. Although most large pharmaceutical companies abandoned the therapeutic strategy of targeting autophagy for cancer treatment, there is a resurgent interest in the field as there is mounting preclinical evidence that inhibiting autophagy can enhance the efficacy of currently used cancer therapies. In fact, the FDA-approved anti-malarial drug, hydroxychloroquine (HCQ), has been pushed into many clinical trials in combination with chemotherapy for various tumor types, including pancreatic cancer and colorectal cancer. HCQ has some dose-sensitive effects, at least in-part due to the lysosomal storage disease phenotype it induces in cells, which can limit the amount of drug given.

Similar to HCQ, past studies targeting NPC1 have shown promise in cancer treatment; however, small molecule approaches were vulnerable to unwanted off-target effects and toxicity. The present disclosure is directed to overcoming these and other limitations in the art.

### SUMMARY

A first aspect of the disclosure relates to a Niemann-Pick disease, type C1 (NPC1) binding polypeptide. This NPC1 binding polypeptide comprises a fibronectin type III (FN3) domain having a modified FG loop amino acid sequence, a modified BC loop amino acid sequence, a modified CD loop amino acid sequence, a modified DE loop amino acid sequence, or a combination thereof, wherein said one or more modified loop sequences enable binding to NPC1.

Another aspect of the present disclosure relates to a NPC1 binding peptide conjugate. This NPC1 binding peptide conjugate comprises a first portion and a second portion. The first portion of the NPC1 binding peptide conjugate comprises the NPC1 binding polypeptide as described herein, and the second portion of the conjugate, which is coupled to the first portion, is selected from a pharmaceutically active moiety, a diagnostic moiety, a half-life extending moiety, a delivery vehicle, a prodrug, a second binding molecule, a polymer, and a non-binding protein.

Other aspects of the present disclosure relate to isolated polynucleotides encoding the NPC1 binding polypeptides as described herein, isolated polynucleotides encoding the NPC1 binding peptide conjugate as described herein, and a vector comprising any one of the described polynucleotides. Another aspect of the present disclosure relates to host cells containing these polynucleotides or vectors.

Another aspect of the present disclosure relates to a pharmaceutical composition comprising the NPC1 binding polypeptide as described herein, the NPC1 binding peptide conjugate as described herein, the isolated polynucleotide as described herein, or the vector as described herein, and a pharmaceutical carrier.

Another aspect of the present disclosure relates to a combination therapeutic. This combination therapeutic includes the NPC1 binding polypeptide as described herein and a cancer therapeutic.

Another aspect of the present disclosure relates to a method of treating cancer in a subject. This method involves administering, to the subject having cancer, the pharmaceutical composition as described herein in an amount effective to treat the cancer.

Another aspect of the present disclosure relates to a method for treating an infectious disease in a subject. This method involves administering, to the subject having the infectious disease, the NCP1 binding polypeptide or the NPC1 binding peptide conjugate as described herein in an amount effective to treat the infectious disease.

Another aspect of the present disclosure is directed to a method of enhancing endosomal release of a pharmaceutically active moiety in a subject in need thereof. This method comprises administering, to the subject, a NPC1 binding peptide conjugate, wherein said peptide conjugate comprises a first and second portion as described herein, where the second portion is the pharmaceutically active moiety.

Another aspect of the present disclosure is directed to a method of enhancing endosomal release of a pharmaceutically active moiety in a subject in need thereof. This method comprises administering, to the subject, a combination therapeutic, where the combination therapeutic comprises the NPC1 binding polypeptide as described herein and the pharmaceutically active moiety.

As disclosed herein, NPC1 inhibition disrupts autophagy in cancer cells. Since autophagy is a mechanism of treatment resistance, NPC1 inhibition can be used to enhance re-sensitize cells to treatment and improve efficacy of cancer therapeutics. Current NPC1 inhibitors are not useful for this purpose because they do not selectively target cancer cells. However, the NPC1 binding molecules and NPC1 binding peptide conjugates described herein are specifically internalized by macropinocytosis into endosomal compartments. Macropinocytosis is a process that grants cells the ability to internalize large amounts of extracellular fluid and solutes to support metabolic demands, and is a process that is specifically enhanced in cancers that are driven by mutant Ras, deregulated growth factor signaling, Src activation, and the like. Thus, macropinocytosis mediated uptake of the NPC1 binding molecules and NPC1 binding peptide conjugates described herein provides both a stand-alone cancer therapy, i.e., a means to achieve selective delivery of a cancer therapeutic to cancer cells, and an adjuvant therapy to re-sensitize cancer cells to treatment with a cancer therapeutic.

### BRIEF DESCRIPTION OF THE DRAWINGS

FIGS. 1A-1B show NPC1 expression in cancer. FIG. 1A show NPC1 expression is increased in pancreatic cancer tissue versus normal adjacent tissue. FIG. 1B is a Kaplan-Meier survival analysis showing NPC1 is poor prognosis indicator in pancreatic cancer. Data derived from TCGA datasets.

FIG. 2 shows the effect of NPC1 inhibition on DLD-1 cancer cell proliferation. Proliferation was analyzed by Syto60 assay after 3 days of treatment; n=3.

FIG. 3A shows endosomal accumulation of free cholesterol upon NPC1 knockdown in DLD-1 and HCT-116 cancer cell lines. Filipin labels free cholesterol. FIG. 3B shows inhibition of autophagic flux upon NPC1 knockdown, as indicated by LC3B accumulation. FIG. 3C shows validation of LC3B accumulation by western blot analysis using tool compounds to inhibit NPC1.

FIG. 4 is a schematic showing NPC1 topology. A monobody library was screened for binders of NTD (cholesterol binding) domain in yellow.

FIGS. 5A-5B show NPC1 N-terminal domain (NTD) and C-terminal domain (CTD) binding monobodies. Binding affinities (arbitrary units) for monobody clones that bind NPC1 NTD in FIG. 5A and CTD in FIG. 5B. FC is control for non-specific binding.

FIG. 6 shows NPC1 NTD- and CTD-binding monobodies in cholesterol loaded versus unloaded state. Binding affinities (arbitrary units) for monobody clones that bind NPC1 NTD (left) and CTD (right). FC is control for non-specific binding.

FIGS. 7A-7C show the results of a screen for NPC1-inhibitory monobodies.

FIG. 7A is a graph showing the effect of monobody clones on intracellular cholesterol trafficking. FIG. 7B are representative cell images from FIG. 7A. FIG. 7C is a heat map representation of cholesterol localization from FIG. 7B.

FIGS. 8A-8B show mutant KRas-dependent effects of NPC1-targeting monobodies. N23 and N34 clones were analyzed for effect on macropinocytosis negative wild-type KRas HeLa cells (FIG. 8A) versus macropinocytosis-positive mutant KRas HeLa cells (FIG. 8B). FN is a non-targeting monobody control. Arrows indicate LC3B accumulation.

FIG. 9 shows the effect of monobody candidates on HCT-116 cell proliferation. Proliferation was analyzed by Syto60 assay after 3 days of treatment. n=3

FIGS. 10A-10B show monobody selectivity in colorectal DLD-1 and HCT-116 cancer cells (CRC). Candidate monobody N34 shows selective uptake (FIG. 10A) and biological effect (FIG. 10B) in mutant KRas CRC cell lines.

FIG. 11 show the in vivo cholesterol alterations with NPC1-targeting monobody (N34) versus non-targeting control (FN).

FIG. 12 shows the in vivo biological effect of NPC1-targeting monobody. Candidate monobody N34 induces cholesterol and LC3B accumulation in N34-positive tumor versus N34-negative tumor. Monobody (1 uM; 50ul volume) was intratumorally injected two hours prior to tumor extraction.

FIGS. 13A-13B show that ERK hyperactivation occurs following NPC1 inhibition in vitro and in vivo. FIG. 13A shows NPC1 knockdown in DLD-1 and HCT-116 cell lines results in increased ERK activation. Candidate monobody N34 induces ERK phosphorylation in N34-positive tumor versus N34-negative tumor as shown in FIG. 13B. Monobody (1 uM; 50ul volume) was intratumorally injected two hours prior to tumor extraction.

FIG. 14 shows ERK hyperactivation is driven by EGFR signaling. ERK hyperactivation following NPC1 knockdown can be reversed upon short-term EGFR inhibition by dacomitinib.

FIG. 15 shows EGFR phosphorylation following NPC1-targeting monobody treatment. Candidate monobody N34 induces EGFR phosphorylation in N34-positive tumor versus N34-negative tumor. Monobody (1 uM; 50ul volume) was intratumorally injected two hours prior to tumor extraction. Images were taken of serial sections from FIG. 13B.

FIG. 16 shows NCP1 monobody induced endosomal release of GFP11 using a split GFP assay. Mutant Ras PDAC MIA PaCa-2 cells stably expressing cytoplasmic GFP1-10 were treated with 600 mM GFP11 with or without 1 mM of the N23 or N34 NCP1 monobody for 24 hrs. Fluorescence is dependent on endosomal escape of GFP11, which was observed in cells treated with the NCP1 monobodies, but not the non-binding FN monobody.

FIG. 17 shows NCP1 monobody induced endosomal release of calcein. Calcein is a membrane impermeable, fluid phase uptake marker that is semi-quenched when in close proximity with other calcein molecules in vesicular compartments, but with intracellular release and molecule diffusion, dequenching causes an increase in cellular fluorescence. FIG. 17 shows increasing calcein fluorescence with N23 and N34 NCP1 monobody treatment, but not treatment with the non-binding FN monobody.

FIGS. 18A-18B show an NCP1 monobody mediated increase in endosomal calcein release that was further improved in the presence of a nanoparticle delivery vehicle. FIG. 18A is a panel of immunocytochemical images of PDAC MIA PaCa3 cells treated with calcein alone (PBS) or packaged in a pegylated nanoparticle delivery vehicles (90 nm Nano) (images of top row). Co-treatment of the cells with the N23 or N34 NCP1 monobodies, respectively, enhanced endosomal release of calcein under both conditions. FIG. 18B is a graph quantifying calcein fluorescence in each of the tested conditions. The highest level of calcein fluorescence was observed in cells treated with nanoparticles containing calcein and a NPC1 monobody.

### DETAILED DESCRIPTION

The present invention relates generally to Niemann-Pick disease, type C1 (NPC1) binding polypeptides and NPC1 binding peptide conjugates comprising these binding polypeptides and methods of using these NPC1 binding polypeptides and NPC1 binding peptide conjugates for the treatment of cancer, infectious disease, and other conditions.

Accordingly, a first aspect of the disclosure relates to a Niemann-Pick disease, type C1 (NPC1) binding polypeptide. This NPC1 binding polypeptide comprises a fibronectin type III (FN3) domain having a modified FG loop amino acid sequence, a modified BC loop amino acid sequence, a modified CD loop amino acid sequence, a modified DE loop amino acid sequence, or any combination of the aforementioned modified loop sequences. The one or more modified loop sequences enable binding to NPC1.

The FN3 domain is an evolutionary conserved protein domain that is about 100 amino acids in length and possesses a beta sandwich structure. The beta sandwich structure of human FN3 comprises seven beta-strands, referred to as strands A, B, C, D, E, F, G, with six connecting loops, referred to as loops AB, BC, CD, DE, EF, and FG that exhibit structural homology to immunoglobulin binding domains. Three of the six loops, i.e., loops DE, BC, and FG, correspond topologically to the complementarity determining regions of an antibody, i.e., CDR1, CDR2, and CDR3. The remaining three loops are surface exposed in a manner similar to antibody CDR3. In accordance with the present disclosure, one or more of the loop regions of each FN3 domain of the binding molecule are modified to enable specific binding to NPC1.

As used herein “specifically binds” or “specific binding” refers to the ability of the FN3 containing binding molecule of the disclosure to bind to a predetermined antigen, i.e., a NPC1 with a dissociation constant (K_{D}) of about 1×10⁻⁶ M or less, for example about 1×10⁻⁷ M or less, about 1×10⁻⁸M or less, about 1×10⁻⁹ M or less, about 1×10⁻¹⁰M or less, about 1×10⁻¹¹ M or less, about 1×10⁻¹² M or less, or about 1×10⁻¹³ M or less. Typically, the FN3 domain binds to NPC1 with a K_{D} that is at least ten fold less than its K_{D} for a nonspecific antigen (for example BSA or casein) as measured by surface plasmon resonance using for example a Proteon Instrument (BioRad).

The modified FN3 domain of the binding molecule of the present disclosure can be a FN3 domain derived from any of the wide variety of animal, yeast, plant, and bacterial extracellular proteins containing these domains. In one embodiment, the FN3 domain is derived from a mammalian FN3 domain. Exemplary FN3 domains include, for example and without limitation, any one of the 15 different FN3 domains present in human tenascin C, or the 15 different FN3 domains present in human fibronectin (FN), for example, the 10^{th} fibronectin type III domain. Exemplary FN3 domains also include non-natural synthetic FN3 domains, such as those described in U.S. Pat. Publ. No. 2010/0216708 to Jacobs et al., which is hereby incorporated by reference in its entirety. Individual FN3 domains are referred to by domain number and protein name, e.g., the 10^{th} FN3 domain of fibronectin (10FN3).

In some embodiments, the FN3 domain of the binding molecule is derived from the 10^{th} FN domain of fibronectin (10FN3). In some embodiments, the FN3 domain of the binding molecule is derived from the human 10FN3 domain. The human 10FN3 domain has the amino acid sequence of SEQ ID NO:1 as shown below. The locations of the BC (residues 24-30), CD (residues 40-45), DE (residues 51-55), and FG (residues 75-86) loops are underlined within the wild-type sequence of SEQ ID NO: 1. Locations of other amino acid residues referenced in this disclosure are also identified within SEQ ID NO: 1 by their position.

(SEQ ID NO: 1)
VSDVPRDLEVVAATPTSLLISWDA₂₄PAVTVR₃₀YYR₃₃
ITYGETG₄₀GNSPV₄₅QE₄₇FT₄₉VP₅₁GSK₅₄S₅₅TATIS
GLKPGVDYTITVYA₇₄V₇₅TGRGDSPASSK₈₆PISINYRT

In accordance with the present disclosure, one or more of the loop regions or selected residues within one or more of these loop regions are modified to enable NPC1 binding specificity and affinity. Suitable modifications include amino acid residue substitutions, insertions, and/or deletions. In one aspect, amino acid residues in at least one, at least two, at least three, at least four, at least five, or all six of the loop regions are altered for NPC1 binding specificity and affinity. In one embodiment, one or more amino acid modifications within the loop regions at or about residues 24-30 (BC loop), 40-45 (CD loop), 51-55 (DE loop), and 75-86 (FG loop) of SEQ ID NO:1 form the NPC1 binding region. In another embodiment, one or more amino acid modification within any one of these loop regions enable NPC1 binding.

In some embodiments, the NPC1 binding molecule of the present disclosure comprises a modified BC loop. In some embodiments, the modified BC loop is selected from any one of the modified BC loops of SEQ ID NOs: 15-21 (see Table 1), or a BC loop having an amino acid sequence having at least 90%, at least 91%, at least 92%, at least 93%, at least 94%, at least 95%, at least 96%, at least 97%, at least 98%, at least 99% sequence identity to any one of the amino acid sequences of SEQ ID NOs: 15-21.

In some embodiments, the NPC1 binding molecule of the present disclosure comprises a modified CD loop. In some embodiments, the modified CD loop is selected from any one of the modified CD loops of SEQ ID NOs: 23-28 (see Table 1), or a CD loop having an amino acid sequence having at least 90%, at least 91%, at least 92%, at least 93%, at least 94%, at least 95%, at least 96%, at least 97%, at least 98%, at least 99% sequence identity to any one of the amino acid sequences of SEQ ID NOs: 23-28.

In some embodiments, the NPC1 binding molecule of the present disclosure comprises a modified DE loop. In some embodiments, the modified DE loop comprises the amino acid sequence of SEQ ID NO: 30 (see Table 1), or a DE loop having an amino acid sequence having at least 90%, at least 91%, at least 92%, at least 93%, at least 94%, at least 95%, at least 9600, at least 9700, at least 98%, at least 9900 sequence identity to the amino acid sequences of SEQ TD NO: 30.

In some embodiments, the NPC1 binding molecule of the present disclosure comprises a modified FG loop. In some embodiments, the modified FG loop is selected from any one of the modified FG loops of SEQ TD NOs: 2-13 (see Table 1), or a FG loop having an amino acid sequence having at least 90%, at least 91%, at least 92%, at least 93%, at least 94%, at least 9500 at least 96%, at least 9700 at least 98%, at least 9900 sequence identity to any one of the amino acid sequences of SEQ TD NOs: 2-13.

TABLE 1
Amino Acid Sequences of BC, CD, DE, and
FG Loops of NCP1 Binding Molecules

| | | SEQ | | SEQ | | SEQ | | SEQ |
|---|---|---|---|---|---|---|---|---|
| | BC Loop | ID | CD Loop | ID | DE Loop | ID | FG Loop | ID |
| Name | Sequence | NO: | Sequence | NO: | Sequence | NO: | Sequence | NO: |
| NPC1N- | AYVSYWKVR | 15 | GGNSPV | 29 | PSSSS | 30 | KMYSYPYWYYS | 2 |
| N8 | | | | | | | | |
| NPC1N- | ARGVPIWWQVY | 16 | GGNSPV | 29 | PGSSS | 30 | WGGWKWYS | 3 |
| N16 | | | | | | | | |
| NPC1N- | AQPMYRSVS | 17 | GGNSPV | 29 | PGSSY | 30 | YSYYKGWYWS | 4 |
| N18 | | | | | | | | |
| NPC1N- | APAVTVS | 18 | GGPFWHY | 23 | PGSKS | 31 | YSPYYPAPYRSS | 5 |
| N22 | | | | | | | | |
| NPC1N- | ASSSSVS | 19 | GGPFWHY | 23 | PGSKS | 31 | YSSSMHFSSS | 6 |
| N23 | | | | | | | | |
| NPC1N- | APAVTVS | 18 | GGSYWHY | 24 | PGSKS | 31 | YSPYYPAPYRSS | 7 |
| N24 | | | | | | | | |
| NPC1N- | APAVTVS | 18 | GSPYWHY | 25 | PGSKS | 31 | YSPYYPAPYRSS | 8 |
| N26 | | | | | | | | |
| NPC1N- | APAVTVS | 18 | GGHYWHW | 26 | PGSKS | 31 | KSYMYGPPSYKSS | 9 |
| N31 | | | | | | | | |
| NPC1N- | APAVTVS | 18 | GGHYWHW | 26 | PGSKS | 31 | YYGQMRYYS | 10 |
| N34 | | | | | | | | |
| NPC1N- | APAVTVV | 20 | GGSYWHY | 24 | PGSKS | 31 | YYSSYRYWS | 11 |
| N35 | | | | | | | | |
| NPC1N- | APAVTVD | 21 | GAPVWHV | 27 | PGSKS | 31 | SSSSSGSSSSSK | 12 |
| N38 | | | | | | | | |
| NPC1C- | APAVTVV | 20 | GASPYYY | 28 | PGSKS | 31 | YDGFYYTNNDS | 13 |
| C45 | | | | | | | | |

As discussed above, FN3 domains contain two sets of CDR-like loops on the opposite faces of the molecule. The two sets of loops are separated by beta-strands (regions of the domain that are between the loops) that form the center of the FN3 structure. Like the loops, these beta-strands can be altered to enhance target molecule binding specificity and affinity. Preferably, some or all of the surface exposed residues in the beta strands are randomized without affecting (or minimally affecting) the inherent stability of the FN3 domain. In some embodiments, one or more of residues in one or more beta-strands is modified to enable interaction with NPC1. Suitable modifications include amino acid substitutions, insertions, and/or deletions. For example, one or more amino acid residues of the A beta strand, the B beta strand, the C beta strand, the D beta strand, the E beta strand, the F beta strand, or the G beta strand may be modified to enable NPC1 binding or to enhance the specificity or affinity of NPC1 binding. In one embodiment, one or more amino acid residues of the A, B, C, D, E, and/or F beta-strands are modified for binding to a NPC1.

In some embodiments, the NCP1 binding polypeptide described herein comprises one or more amino acid residue substitutions, additions, or deletions in the A beta strand or region upstream thereof. In some embodiments, the NCP1 binding polypeptide comprises an amino acid substitution at one or more resides corresponding to residues D3, R6 and D7 of SEQ ID NO: 1. In some embodiments, the amino acid substitution is an aspartic acid to serine substitution at the amino acid residue corresponding to the aspartic acid at position 3 (D3S) of SEQ ID NO: 1, an arginine to threonine substitution at the amino acid residue corresponding to the arginine at position 6 (R6T) of SEQ ID NO:1, and/or an aspartic acid to lysine substitution at the amino acid residue corresponding to the aspartic acid at position 7 (D7K) of SEQ ID NO: 1. In some embodiments, the NCP1 binding polypeptide comprises the amino acid substitutions of aspartic acid to serine, arginine to threonine, and aspartic acid to lysine at the amino acid residues corresponding to D3S, R6T, and D7K of SEQ ID NO: 1.

In some embodiments, the NCP1 binding polypeptide described herein comprises one or more amino acid residue substitutions, additions, or deletions in the C beta strand. In some embodiments, the NCP1 binding polypeptide comprises an amino acid substitution in the C beta strand at the residue corresponding to tyrosine residue at position 31 of SEQ ID NO: 1. In some embodiments, the amino acid substitution is a tyrosine to histidine substitution at the amino acid residue corresponding to the tyrosine at position 31 (Y31H) of SEQ ID NO: 1. In some embodiments, the NCP1 binding polypeptide comprises an amino acid substitution in the C beta strand at the residue corresponding to arginine residue at position 33 SEQ ID NO: 1. In some embodiments, the amino acid substitution is an arginine to valine substitution at the amino acid residue corresponding to the arginine at position 33 (R33V) of SEQ ID NO: 1. In some embodiments, the amino acid substitution is an arginine to aspartic acid substitution at the amino acid residue corresponding to the arginine at position 33 (R33D) of SEQ ID NO:1. In some embodiments, the amino acid substitution is an arginine to phenylalanine substitution at the amino acid residue corresponding to the arginine at position 33 (R33F) of SEQ ID NO: 1.

In some embodiments, the NCP1 binding polypeptide described herein comprises one or more amino acid residue substitutions, additions, or deletions in the D beta strand. In some embodiments, the NCP1 binding polypeptide comprises an amino acid substitution in the D beta strand at the residue corresponding to glutamic acid residue at position 47 SEQ ID NO: 1. In some embodiments, the amino acid substitution is a glutamic acid to threonine substitution at the amino acid residue corresponding to the glutamic acid at position 47 (E47T) of SEQ ID NO: 1. In some embodiments, the amino acid substitution is a glutamic acid to lysine substitution at the amino acid residue corresponding to the glutamic acid at position 47 (E47K) of SEQ ID NO:1. In some embodiments, the NCP1 binding polypeptide comprises an amino acid substitution in the D beta strand at the residue corresponding to threonine residue at position 49 SEQ ID NO: 1. In some embodiments, the amino acid substitution is a threonine to lysine substitution at the amino acid residue corresponding to the threonine at position 49 (T49K) of SEQ ID NO: 1. In some embodiments, the amino acid substitution is a threonine to alanine substitution at the amino acid residue corresponding to the threonine at position 49 (T49A) of SEQ ID NO:1.

In some embodiments, the NCP1 binding polypeptide described herein comprises one or more amino acid residue substitutions, additions, or deletions in the F beta strand. In some embodiments, the NCP1 binding polypeptide comprises an amino acid substitution in the D beta strand at the residue corresponding to alanine residue at position 74 SEQ ID NO: 1. In some embodiments, the amino acid substitution is an alanine to threonine substitution at the amino acid residue corresponding to the alanine at position 74 (A74T) of SEQ ID NO: 1.

In some embodiments, the NCP1 binding polypeptide described herein comprises one or more amino acid residue substitutions, additions, or deletions in the A strand, C strand, D strand, E strand, and F beta strand. In some embodiments, the NCP1 binding polypeptide described herein comprises amino acid substitution at positions corresponding to all of the aforementioned amino acid residues, i.e., at residues corresponding to residues D3, R6, D7, Y31, R33, E47, T49, and A74 of SEQ ID NO: 1.

In some embodiments, the NCP1 binding polypeptide as described herein comprises an FN3 domain comprising a modified FG loop amino acid sequence of SEQ ID NO: 2, a modified BC loop amino acid sequence of SEQ ID NO: 15, and a modified DE loop amino acid sequence of SEQ ID NO: 30. In some embodiments, the FN domain further comprises an amino acid substitution at one or more residues corresponding to residues D3, R6, D7, Y31, R33, E47, T49, and A74 of SEQ ID NO: 1. In some embodiments, the FN domain comprises amino acid substitutions at the residues corresponding to residue D3, R6, and D7. In some embodiments, the FN3 domain comprises an amino acid sequence that is at least 80% identical to an amino acid sequence of SEQ ID NO: 32. In some embodiments, the FN3 domain comprises an amino acid sequence that is at least 90% identical to an amino acid sequence of SEQ ID NO: 32. In some embodiments, the FN3 domain comprises an amino acid sequence of SEQ ID NO: 32 (monobody (Mb)NPC1N-N8).

Mb(NPCIN-N8)
(SEQ ID NO: 32)
VSSVPTKLEVVAATPTSLLISWDAYVSYWKVRYYRITYGE
TGGNSPVQEFTVPSSSSTATISGLKPGVDYTITVYAKMYS
YPYWYYSPISINYRT

In some embodiments, the NCP1 binding polypeptide as described herein comprises an FN3 domain comprising a modified FG loop amino acid sequence of SEQ ID NO: 3, a modified BC loop amino acid sequence of SEQ ID NO: 16, and a modified DE loop amino acid sequence of SEQ ID NO: 30. In some embodiments, the FN domain further comprises an amino acid substitution at one or more residues corresponding to residues D3, R6, D7, Y31, R33, E47, T49, and A74 of SEQ ID NO: 1. In some embodiments, the FN domain comprises amino acid substitutions at the residues corresponding to residue D3, R6, and D7. In some embodiments, the FN3 domain comprises an amino acid sequence that is at least 80% identical to an amino acid sequence of SEQ ID NO: 33. In some embodiments, the FN3 domain comprises an amino acid sequence that is at least 90% identical to an amino acid sequence of SEQ ID NO: 33. In some embodiments, the FN3 domain comprises an amino acid sequence of SEQ ID NO: 33 (MbNPC1N-N16).

Mb(NPCIN-N16)
(SEQ ID NO: 33)
VSSVPTKLEVVAATPTSLLISWDARGVPIWWQVYYYRITY
GETGGNSPVQEFTVPGSSSTATISGLKPGVDYTITVYAWG
GWKWYSPISINYRT

In some embodiments, the NCP1 binding polypeptide as described herein comprises an FN3 domain comprising a modified FG loop amino acid sequence of SEQ ID NO: 4, a modified BC loop amino acid sequence of SEQ ID NO: 17, and a modified DE loop amino acid sequence of SEQ ID NO: 30. In some embodiments, the FN domain further comprises an amino acid substitution at one or more residues corresponding to residues D3, R6, D7, Y31, R33, E47, T49, and A74 of SEQ ID NO: 1. In some embodiments, the FN domain comprises amino acid substitutions at the residues corresponding to residue D3, R6, and D7. In some embodiments, the FN3 domain comprises an amino acid sequence that is at least 80% identical to an amino acid sequence of SEQ ID NO: 34. In some embodiments, the FN3 domain comprises an amino acid sequence that is at least 90% identical to an amino acid sequence of SEQ ID NO: 34. In some embodiments, the FN3 domain comprises an amino acid sequence of SEQ ID NO: 34 (MbNPC1N-N18).

Mb(NPCIN-N18)
(SEQ ID NO: 34)
VSSVPTKLEVVAATPTSLLISWDAQPMYRSVSYYRITYGE
TGGNSPVQEFTVPGSSYTATISGLKPGVDYTITVYAYSYY
KGWYWSPISINYRT

In some embodiments, the NCP1 binding polypeptide as described herein comprises an FN3 domain comprising a modified FG loop amino acid sequence of SEQ ID NO: 5, a modified BC loop amino acid sequence of SEQ ID NO: 18, and a modified CD loop amino acid sequence of SEQ ID NO: 23. In some embodiments, the FN domain further comprises an amino acid substitution at one or more residues corresponding to residues D3, R6, D7, Y31, R33, E47, T49, and A74 of SEQ ID NO: 1. In some embodiments, the FN domain comprises amino acid substitutions at the residues corresponding to residue D3, R6, D7, R33, and E47. In some embodiments, the FN3 domain comprises an amino acid sequence that is at least 80% identical to an amino acid sequence of SEQ ID NO: 35. In some embodiments, the FN3 domain comprises an amino acid sequence that is at least 90% identical to an amino acid sequence of SEQ ID NO: 35. In some embodiments, the FN3 domain comprises an amino acid sequence of SEQ ID NO: 35 (MbNPC1N-N22).

Mb(NPCIN-N22)
(SEQ ID NO: 35)
VSSVPTKLEVVAATPTSLLISWDAPAVTVSYYVITYGETG
GPFWHYQTFTVPGSKSTATISGLKPGVDYTITVYAYSPYY
PAPYRSSPISINYRT

In some embodiments, the NCP1 binding polypeptide as described herein comprises an FN3 domain comprising a modified FG loop amino acid sequence of SEQ ID NO: 6, a modified BC loop amino acid sequence of SEQ ID NO: 19, and a modified CD loop amino acid sequence of SEQ ID NO: 23. In some embodiments, the FN domain further comprises an amino acid substitution at one or more residues corresponding to residues D3, R6, D7, Y31, R33, E47, T49, and A74 of SEQ ID NO: 1. In some embodiments, the FN domain comprises amino acid substitutions at the residues corresponding to residue D3, R6, D7, E47, and A74. In some embodiments, the FN3 domain comprises an amino acid sequence that is at least 80% identical to an amino acid sequence of SEQ ID NO: 36. In some embodiments, the FN3 domain comprises an amino acid sequence that is at least 90% identical to an amino acid sequence of SEQ ID NO: 36. In some embodiments, the FN3 domain comprises an amino acid sequence of SEQ ID NO: 36 (MbNPC1N-N23).

Mb(NPC1N-N23)
(SEQ ID NO: 36)
VSSVPTKLEVVAATPTSLLISWDASSSSVSYYRITYGETG
GPFWHYQTFTVPGSKSTATISGLKPGVDYTITVYTYSSSM
HFSSSPISINYRT

In some embodiments, the NCP1 binding polypeptide as described herein comprises an FN3 domain comprising a modified FG loop amino acid sequence of SEQ ID NO: 7, a modified BC loop amino acid sequence of SEQ ID NO: 18, and a modified CD loop amino acid sequence of SEQ ID NO: 24. In some embodiments, the FN domain further comprises an amino acid substitution at one or more residues corresponding to residues D3, R6, D7, Y31, R33, E47, T49, and A74 of SEQ ID NO: 1. In some embodiments, the FN domain comprises amino acid substitutions at the residues corresponding to residue D3, R6, D7, R33, E47, and T49. In some embodiments, the FN3 domain comprises an amino acid sequence that is at least 80% identical to an amino acid sequence of SEQ ID NO: 37. In some embodiments, the FN3 domain comprises an amino acid sequence that is at least 90% identical to an amino acid sequence of SEQ ID NO: 37. In some embodiments, the FN3 domain comprises an amino acid sequence of SEQ ID NO: 37 (MbNPC1N-N24).

Mb(NPCIN-N24)
(SEQ ID NO: 37)
VSSVPTKLEVVAATPTSLLISWDAPAVTVSYYVITYGETG
GSYWHYQTFKVPGSKSTATISGLKPGVDYTITVYAYSPYY
PAPYRSSPISINYRT

In some embodiments, the NCP1 binding polypeptide as described herein comprises an FN3 domain comprising a modified FG loop amino acid sequence of SEQ ID NO: 8, a modified BC loop amino acid sequence of SEQ ID NO: 18, and a modified CD loop amino acid sequence of SEQ ID NO: 25. In some embodiments, the FN domain further comprises an amino acid substitution at one or more residues corresponding to residues D3, R6, D7, Y31, R33, E47, T49, and A74 of SEQ ID NO: 1. In some embodiments, the FN domain comprises amino acid substitutions at the residues corresponding to residue D3, R6, D7, R33, and E47. In some embodiments, the FN3 domain comprises an amino acid sequence that is at least 80% identical to an amino acid sequence of SEQ ID NO: 38. In some embodiments, the FN3 domain comprises an amino acid sequence that is at least 90% identical to an amino acid sequence of SEQ ID NO: 38. In some embodiments, the FN3 domain comprises an amino acid sequence of SEQ ID NO: 38 (MbNPC1N-N26).

Mb(NPC1N-N26)
(SEQ ID NO: 38)
VSSVPTKLEVVAATPTSLLISWDAPAVTVSYYVITYGETG
SPYWHYQTFTVPGSKSTATISGLKPGVDYTITVYAYSPYY
PAPYRSSPISINYRT

In some embodiments, the NCP1 binding polypeptide as described herein comprises an FN3 domain comprising a modified FG loop amino acid sequence of SEQ ID NO: 9, a modified BC loop amino acid sequence of SEQ ID NO: 18, and a modified CD loop amino acid sequence of SEQ ID NO: 26. In some embodiments, the FN domain further comprises an amino acid substitution at one or more residues corresponding to residues D3, R6, D7, Y31, R33, E47, T49, and A74 of SEQ ID NO: 1. In some embodiments, the FN domain comprises amino acid substitutions at the residues corresponding to residue D3, R6, D7, R33, and E47. In some embodiments, the FN3 domain comprises an amino acid sequence that is at least 80% identical to an amino acid sequence of SEQ ID NO: 39. In some embodiments, the FN3 domain comprises an amino acid sequence that is at least 90% identical to an amino acid sequence of SEQ ID NO: 39. In some embodiments, the FN3 domain comprises an amino acid sequence of SEQ ID NO: 39 (MbNPC1N-N31).

Mb(NPCIN-N31)
(SEQ ID NO: 39)
VSSVPTKLEVVAATPTSLLISWDAPAVTVSYYVITYGETG
GHYWHWQTFTVPGSKSTATISGLKPGVDYTITVYAKSYMY
GPPSYKSSPISINYRT

In some embodiments, the NCP1 binding polypeptide as described herein comprises an FN3 domain comprising a modified FG loop amino acid sequence of SEQ ID NO: 10, a modified BC loop amino acid sequence of SEQ ID NO: 18, and a modified CD loop amino acid sequence of SEQ ID NO: 26. In some embodiments, the FN domain further comprises an amino acid substitution at one or more residues corresponding to residues D3, R6, D7, Y31, R33, E47, T49, and A74 of SEQ ID NO: 1. In some embodiments, the FN domain comprises amino acid substitutions at the residues corresponding to residue D3, R6, D7, R33, E47, and T49. In some embodiments, the FN3 domain comprises an amino acid sequence that is at least 80% identical to an amino acid sequence of SEQ ID NO: 40. In some embodiments, the FN3 domain comprises an amino acid sequence that is at least 90% identical to an amino acid sequence of SEQ ID NO: 40. In some embodiments, the FN3 domain comprises an amino acid sequence of SEQ ID NO: 40 (MbNPC1N-N34).

(SEQ ID NO: 40)
Mb(NPCIN-N34)
VSSVPTKLEVVAATPTSLLISWDAPAVTVSYYVITYGETG
GHYWHWQTFKVPGSKSTATISGLKPGVDYTITVYAYYGQM
RYYSPISINYRT

In some embodiments, the NCP1 binding polypeptide as described herein comprises an FN3 domain comprising a modified FG loop amino acid sequence of SEQ ID NO: 11, a modified BC loop amino acid sequence of SEQ ID NO: 20, and a modified CD loop amino acid sequence of SEQ ID NO: 24. In some embodiments, the FN domain further comprises an amino acid substitution at one or more residues corresponding to residues D3, R6, D7, Y31, R33, E47, T49, and A74 of SEQ ID NO: 1. In some embodiments, the FN domain comprises amino acid substitutions at the residues corresponding to residue D3, R6, D7, R33, E47, and T49. In some embodiments, the FN3 domain comprises an amino acid sequence that is at least 80% identical to an amino acid sequence of SEQ ID NO: 41. In some embodiments, the FN3 domain comprises an amino acid sequence that is at least 90% identical to an amino acid sequence of SEQ ID NO: 41. In some embodiments, the FN3 domain comprises an amino acid sequence of SEQ ID NO: 41 (MbNPC1N-N35).

Mb(NPCIN-N35)
(SEQ ID NO: 41)
VSSVPTKLEVVAATPTSLLISWDAPAVTVVYYDITYGETG
GSYWHYQTFKVPGSKSTATISGLKPGVDYTITVYAYYSSY
RYWSPISINYRT

In some embodiments, the NCP1 binding polypeptide as described herein comprises an FN3 domain comprising a modified FG loop amino acid sequence of SEQ ID NO: 12, a modified BC loop amino acid sequence of SEQ ID NO: 21, and a modified CD loop amino acid sequence of SEQ ID NO: 27. In some embodiments, the FN domain further comprises an amino acid substitution at one or more residues corresponding to residues D3, R6, D7, Y31, R33, E47, T49, and A74 of SEQ ID NO: 1. In some embodiments, the FN domain comprises amino acid substitutions at the residues corresponding to residue D3, R6, D7, Y31, R33, and E47. In some embodiments, the FN3 domain comprises an amino acid sequence that is at least 80% identical to an amino acid sequence of SEQ ID NO: 42. In some embodiments, the FN3 domain comprises an amino acid sequence that is at least 90% identical to an amino acid sequence of SEQ ID NO: 42. In some embodiments, the FN3 domain comprises an amino acid sequence of SEQ ID NO: 42 (MbNPC1N-N38).

Mb(NPC1N-N38)
(SEQ ID NO: 42)
VSSVPTKLEVVAATPTSLLISWDAPAVTVDHYFITYGETG
APVWHVQKFTVPGSKSTATISGLKPGVDYTITVYASSSSS
GSSSSSKPISINYRT

In some embodiments, the NCP1 binding polypeptide as described herein comprises an FN3 domain comprising a modified FG loop amino acid sequence of SEQ ID NO: 13, a modified BC loop amino acid sequence of SEQ ID NO: 20, and a modified CD loop amino acid sequence of SEQ ID NO: 28. In some embodiments, the FN domain further comprises an amino acid substitution at one or more residues corresponding to residues D3, R6, D7, Y31, R33, E47, T49, and A74 of SEQ ID NO: 1. In some embodiments, the FN domain comprises amino acid substitutions at the residues corresponding to residue D3, R6, D7, Y31, R33, E47, and T49. In some embodiments, the FN3 domain comprises an amino acid sequence that is at least 80% identical to an amino acid sequence of SEQ ID NO: 43. In some embodiments, the FN3 domain comprises an amino acid sequence that is at least 90% identical to an amino acid sequence of SEQ ID NO: 43. In some embodiments, the FN3 domain comprises an amino acid sequence of SEQ ID NO: 43 (MbNPC1C-C45).

Mb(NPC1C-C45)
(SEQ ID NO: 43)
VSSVPTKLEVVAATPTSLLISWDAPAVTVVHYVITYGETG
ASPYYYQKFAVPGSKSTATISGLKPGVDYTITVYAYDGFY
YTNNDSPISINYRT

Another aspect of the present disclosure relates to a NP 1 binding peptide conjugate comprising a first portion and a second portion. The first portion of the NPC1 binding peptide conjugate comprises the NPC1 binding polypeptide as described supra. The second portion of the NPC1 binding peptide conjugate, which is coupled to the first portion of the conjugate, is selected from a pharmaceutically active moiety, a diagnostic moiety, a half-life extending moiety, a prodrug, a second binding molecule, a delivery vehicle, a polymer, a non-binding protein, and any combination thereof.

In accordance with this aspect of the present disclosure, the first and second portions of the NPC1 binding peptide conjugate are covalently coupled to either other directly or via a linker. The first and second portions may be directly fused and generated by standard cloning and expression techniques. Alternatively, well known chemical coupling methods may be used to attach the portions directly or via a peptide or other linker to produce NPC1 binding peptide conjugates as described herein. For example, covalent conjugation of the first and second portions can be accomplished via lysine side chains using an activated ester or isothiocyanate, or via cysteine side chains with a maleimide, haloacetyl derivative or activated disulfide. Site specific conjugation of the first and second portions can also be accomplished by incorporating unnatural amino acids, self-labeling tags (e.g., SNAP or DHFR), or a tag that is recognized and modified specifically by another enzyme such as sortase A, lipoic acid ligase, and formylglycine-generating enzyme. In some embodiments, site specific conjugation of the first and second portions is achieved by the introduction of cysteine residue either at the C-terminus of the NPC1 binding molecule or at a specific site as described by Goldberg et al., “Engineering a Targeted Delivery Platform Using Centyrins,” Protein Engineering, Design & Selection 29(12):563-572 (2016), which is hereby incorporated by reference in its entirety.

In some embodiments, the first and second portions of the NPC1 binding peptide conjugate are coupled together via a linker. In some embodiments, the linker is an amino acid linker. In some embodiments, the amino acid linker is a cleavable linker. In some embodiments, the amino acid linker is a non-cleavable linker. Suitable linkers include peptides composed of repetitive modules of one or more of the amino acids, such as glycine and serine or alanine and proline. Exemplary linker peptides include, e.g., (Gly-Gly)ₙ, (Gly-Ser)ₙ, (Gly₃-Ser)ₙ, (Ala-Pro)ₙ wherein ₙ is an integer from 1-25. The length of the linker may be appropriately adjusted as long as it does not affect the function of the non-binding protein-drug conjugate. The standard 15 amino acid (Gly₄-Ser)₃ linker peptide has been well-characterized and has been shown to adopt an unstructured, flexible conformation. In addition, this linker peptide does not interfere with assembly and activity of the domains it connects (Freund et al., “Characterization of the Linker Peptide of the Single-Chain Fv Fragment of an Antibody by NMR Spectroscopy,” FEBS 320:97 (1993), the disclosure of which is hereby incorporated by reference in its entirety).

In some embodiments, the second portion of the NPC1 binding peptide conjugate of the present disclosure comprises a half-life extending moiety. Exemplary half-life extending moieties include, without limitation, albumin, albumin variants (see e.g., U.S. Pat. No. 8,822,417 to Andersen et al., U.S. Pat. No. 8,314,156 to Desai et al., and U.S. Pat. No. 8,748,380 to Plumridge et al., which are hereby incorporated by reference in their entirety), albumin-binding proteins and/or domains, transferrin and fragments and analogues thereof (see e.g., U.S. Pat. No. 7,176,278 to Prior et al., which are hereby incorporated by reference in their entirety), Fc regions and variant Fc regions (see e.g., U.S. Pat. No. 8,546,543 to Lazar et al., U.S. Patent Publication No. 20150125444 to Tsui, and U.S. Pat. No. 8,722,615 to Seehra et al., which are hereby incorporated by reference in their entirety).

Other second portion half-life extending moieties of the NPC1 binding peptide conjugate include, without limitation, polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. A pegyl moiety may for example be added to the first portion, i.e., the NPC1 binding molecule, by adding a cysteine residue to the C-terminus of the molecule and attaching a pegyl group to the cysteine using methods well known in the art.

In another embodiment, the second portion of the NPC1 binding peptide conjugate comprises a diagnostic moiety. Suitable diagnostic moieties are those that facilitate the detection, quantitation, separation, and/or purification of the NPC1 binding peptide conjugate. Suitable diagnostic moieties include, without limitation, purification tags (e.g., poly-histidine (His₆₋), glutathione-S-transferase (GST-), or maltose-binding protein (MBP-)), fluorescent dyes or tags (e.g., chelates (europium chelates), fluorescein and its derivatives, rhodamine and its derivatives, dansyl, Lissamine, phycoerythrin and Texas Red), an enzymatic tag, a radioisotope or radioactive label (e.g., ⁴C, ¹¹C, ¹⁴N, ³⁵S, ³H, ³²P ^{99m}Tc, ¹¹¹In, ^{62/64}Cu, ¹²⁵I ¹⁸F, ^{67/68}Ga, ⁹⁰Y, ¹⁷⁷Lu and ^{186/188}Re), a radionucleotide with chelator (e.g., MAG3, DTPA, and DOTA, see also, Liu S., “Bifunctional Coupling Agents for Radiolabeling of Biomolecules and Target Specific Delivery of Metallic Radionuclides,” Adv. Drug Deli. 60(12):1347-1370 (2008), which is hereby incorporated by reference in its entirety), a contrast agent suitable for imaging, or a photosensitize.

Suitable chelators to be used in combination with a radionucleotide as a diagnostic moiety include, without limitation, NOTA (1, 4, 7-triaza-cyclononane-N,N′,N″-triacetic acid), DOTA (1, 4, 7, 10-tetraazacyclododecane-1, 4, 7, 10-tetraacetic acid), DTP A (1, 1, 4, 7, 7-Diethylenetriaminepentaacetic acid), TETA (p-bromoacetamido-benzyl-tetraethylaminetetraacetic acid), and Df (desferrioxamine B), each of which can be used with a variety of radiolabels, radionuclides, radioisotopes, metals and radiometals. DOTA-type chelators, where the ligand includes hard base chelating functions such as carboxylate or amine groups, are most effective for chelating hard acid cations. Such metal-chelate complexes can be made very stable by tailoring the ring size to the metal of interest. Also, more than one type of chelator may be conjugated to the targetable construct to bind multiple metal ions, e.g., diagnostic radionuclides and/or therapeutic radionuclides.

Chelators can be covalently bound to the NPC1 binding polypeptide of the conjugate (i.e., the FN3 domain) using standard methods of bioconjugation. Amine containing residues (e.g., lysine) in the FN3 domain undergo amide bond formation with a chelator containing an activated ester (e.g., an N-hydroxysuccinimidyl ester). Sulfur containing residues (e.g., cysteine) undergo conjugation with chelators containing an activated ester or maleimide moiety. Alternatively, bioconjugates are formed when activated carboxylate residues of the FN3 domain undergo amide or thoiester formation with amine or thiol groups, respectively, on the chelator. Bifunctional linkers, such as, for example, PEG-maleimide (PEG-Mal), succinimidyl-4-(N-maleimidomethyl)cyclohexane-1-carboxylate (SMCC) or N-succinimidyl 3-(2-pyridylthio)propionate (SPDP) can be alternatively used.

Suitable imaging agents for use as diagnostic moieties in the NPC1 binding peptide conjugate include, without limitation, single photon emission computed tomography (SPECT) agents, positron emission tomography (PET) agents, magnetic resonance imaging (MRI) agents, nuclear magnetic resonance imaging (NMR) agents, x-ray agents, optical agents (e.g., fluorophores, bioluminescent probes, near infrared dyes, quantum dots), ultrasound agents and neutron capture therapy agents, computer assisted tomography agents, two photon fluorescence microscopy imaging agents, and multi-photon microscopy imaging agents. Particularly useful diagnostic radiolabels, radionuclides, or radioisotopes that can be bound to a chelating agent include, without limitation ¹¹⁰In, ^{m}In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶y 9V, ⁸⁹Zr, ⁹⁴Tc, ⁹⁴Tc, ^{99m}Tc, ¹²⁰I ¹²³I ¹²⁴I ¹²⁵I ¹³¹I ¹⁵⁴Gd, ¹⁵⁸Gd, ³²P ¹C, ¹³N, ¹⁵O ¹⁸⁶Re, ¹⁸⁸Re, ⁵¹Mn, ^{2m}Mn, ⁵⁵Co, ⁷²As, ⁷⁵Br, ⁷⁶Br, ^{82m}Rb, ¹³Sr, or other gamma-, beta-, or positron-emitters and ultra-small superparamagnetic particles of iron oxide (USPIO) which are suitable for MRI. The diagnostic radiolabels include a decay energy in the range of 25 to 10,000 keV, more preferably in the range of 25 to 4,000 keV, and even more preferably in the range of 20 to 1,000 keV, and still more preferably in the range of 70 to 700 keV. Total decay energies of useful positron-emitting radionuclides are preferably <2,000 keV, more preferably under 1,000 keV, and most preferably <700 keV.

In another embodiment, the second portion of the NPC1 binding peptide conjugate comprises a pharmaceutically active moiety. Suitable pharmaceutically active moieties include, without limitation, small molecule active moieties, nucleic acid molecules, antibodies or antigen binding fragments thereof, antibody derivatives, a protein or polypeptide fragment thereof, and a proteolysis targeting chimera (PROTAC).

In some embodiments, the pharmaceutically active moiety of the NPC1 binding peptide conjugate is a cancer therapeutic. Suitable cancer therapeutics include, without limitation, an antimetabolite, an alkaloid, an alkylating agent, an anti-mitotic agent, an antitumor antibiotic, a DNA binding drug, a toxin, an antiproliferative drug, a DNA antagonist, a radionuclide, a thermoablative agent, a proteolysis targeting chimera (PROTAC), and a nucleic acid inhibitor, and an immune-modulatory agent.

In some embodiments, the cancer therapeutic is an alkaloid. Suitable alkaloids include, without limitation, duocarmycin, docetaxel, etoposide, irinotecan, paclitaxel, teniposide, topotecan, vinblastine, vincristine, vindesine and analogs and derivatives thereof.

In some embodiments, the cancer therapeutic is an alkylating agent. Suitable alkylating agents include, without limitation, busulfan, improsulfan, piposulfan, benzodepa, carboquone, meturedepa, uredepa, altretamine, triethylenemelamine, triethylenephosphoramide, triethylenethiophosphorarnide, chlorambucil, chloranaphazine, cyclophosphamide, estramustine, ifosfamide, mechlorethamine, mechlorethamine oxide HCl, melphalan, novemebichin, perfosfamide phenesterine, prednimustine, trofosfamide, uracil mustard, carmustine, chlorozotocin, fotemustine, lomustine, nimustine, semustine ranimustine, dacarbazine, mannomustine, mitobronitol, mitolactol, pipobroman, temozolomide, and analogs and derivatives thereof.

In some embodiments, the cancer therapeutic is an antitumor antibiotic. Suitable antitumor antibiotics include, without limitation, aclacinomycin, actinomycin, anthramycin, azaserine, bleomycin, cactinomycin, calicheamicin, carubicin, carzinophilin, cromomycin, dactinomycin, daunorubicin, 6-diazo-5-oxo-1-norleucine, doxorubicin, epirabicin, idarubicin, menogaril, mitomycin, mycophenolic acid, nogalamycine, olivomycin, peplomycin, pirarubicin, plicamycin, porfiromycin, puromycine, pyrrolobenzodiazepine, streptonigrin, streptozocin, tubercidin, zinostatin, zorubicin, and analogs and derivatives thereof.

In some embodiments, the cancer therapeutic is an antimetabolite agent. Suitable antimetabolite agents include, without limitation, SN-38, denopterin, edatrexate, mercaptopurine (6-MP), methotrexate, piritrexim, pteropterin, pentostatin (2′-DCF), tomudex, trimetrexate, cladridine, fludarabine, thiamiprine, ancitabine, azacitidine, 6-azauridine, carmofur, cytarabine, doxifluridine, emitefur, floxuridine, fluorouracil, gemcitabine, tegafur, hydroxyurea, urethane, and analogs and derivatives thereof.

In some embodiments, the cancer therapeutic is an anti-proliferative drug. Suitable anti-proliferative drugs include, without limitation, aceglatone, amsacrine, bisantrene, camptothecin, defosfamide, demecolcine, diaziquone, diflomotecan, eflornithine, elliptinium acetate, etoglucid, etopside, fenretinide, gallium nitrate, hydroxyurea, lamellarin D, lonidamine, miltefosine, mitoguazone, mitoxantrone, mopidamol, nitracrine, pentostatin, phenamet, podophillinic acid 2-ethyl-hydrazide, procarbazine, razoxane, sobuzoxane, spirogermanium, teniposide, tenuazonic acid, triaziquone 2,2′,2″-trichlorotriethylamine, and analogs and derivatives thereof.

In some embodiments, the cancer therapeutic is an antimitotic agent. Suitable antimitotic agents include, without limitation, auristatin, a maytansinoid, a dolastatin, a tubulysin, a taxane, a epothilone, a vinca alkaloid, and analogs and derivatives thereof.

In some embodiments, the pharmaceutically active moiety of the NPC1 binding peptide conjugate is an immunomodulatory agent. Suitable immunomodulatory agents include, without limitation, a macrophage type-1 stimulating agent, a macrophage type-2 stimulating agent, dendritic cell stimulating agent, a neutrophil stimulating agent, a B cell stimulating agent, a T cell stimulating agent.

In some embodiments, the pharmaceutically active moiety of the NPC1 binding peptide conjugate is an immunomodulatory agent that is a macrophage type-1 stimulating agent. Suitable macrophage type-1 stimulating agents include, without limitation, paclitaxel, a colony stimulating factor −1 (CSF-1) receptor antagonist, an IL-10 receptor antagonist, a Toll-like receptor (TLR)-2 agonist, a TLR-3 agonist, a TLR-4 agonist, a TLR-7 agonist, a TLR-8 agonist, and a TLR-9 agonist, and analogs and derivatives thereof.

In any embodiment, the macrophage type-1 stimulating agent is a CSF-1 receptor antagonist. Suitable CSF-1 receptor antagonists include, without limitation, ABT-869 (Guo et al., “Inhibition of Phosphorylation of the Colony-Stimulating Factor-1 Receptor (c-Fms) Tyrosine Kinase in Transfected Cells by ABT-869 and Other Tyrosine Kinase Inhibitors,” Mol. Cancer. Ther. 5(4):1007-1012 (2006), which is hereby incorporated by reference in its entirety), imatinib (Guo et al., “Inhibition of Phosphorylation of the Colony-Stimulating Factor-1 Receptor (c-Fms) Tyrosine Kinase in Transfected Cells by ABT-869 and Other Tyrosine Kinase Inhibitors,” Mol. Cancer. Ther. 5(4):1007-1012 (2006), which is hereby incorporated by reference in its entirety), PLX3397 (Mok et al., “Inhibition of CSF1 Receptor Improves the Anti-tumor Efficacy of Adoptive Cell Transfer Immunotherapy,” Cancer Res. 74(1):153-161 (2014), which is hereby incorporated by reference in its entirety), PLX5622 (Dagher et al., “Colony-stimulating Factor 1 Receptor Inhibition Prevents Microglial Plaque Association and Improves Cognition in 3xTg-AD Mice,” J. Neuroinflamm. 12:139 (2015), which is hereby incorporated by reference in its entirety), DCC-3014 (Deciphera Pharmaceuticals), BLZ945 (Krauser et al., “Phenotypic and Metabolic Investigation of a CSF-1R Kinase Receptor Inhibitor (BLZ945) and its Pharmacologically Active Metabolite,” Xenobiotica 45(2):107-123 (2015), which is hereby incorporated by reference in its entirety), and GW2580 (Olmos-Alonso et al., “Pharmacological Targeting of CSF1R Inhibits Microglial Proliferation and Prevents the Progression of Alzheimer's-like Pathology,” Brain 139:891-907 (2016), which is hereby incorporated by reference in its entirety.

In any embodiment, the macrophage type-1 stimulating agent is an IL-10 receptor antagonist. Suitable IL-10 receptor antagonists include, without limitation, peptide antagonists as described in Naiyer et al., “Identification and Characterization of a Human IL-10 Receptor Antagonist,” Hum. Immunol. 74(1):28-31 (2013), which is hereby incorporated by reference in its entirety, and IL-10 receptor antagonistic antibodies as described in U.S. Pat. No. 7,553,932 to Von Herrath et al., which is hereby incorporated by reference in its entirety.

In any embodiment, the macrophage type-1 stimulating agent is a TLR-2 agonist. Suitable TLR-2 agonists for use in the methods described herein include Pam3CSK4, a synthetic triacylated lipoprotein, and lipoteichoic acid (LTA) (Brandt et al., “TLR2 Ligands Induce NF-xB Activation from Endosomal Compartments of Human Monocytes” PLoS One 8(12):e80743, which is hereby incorporated by reference in its entirety). A suitable TLR-3 agonist includes, without limitation, polyinosinic:polycytidylic acid (poly I:C) (Smole et al., “Delivery System for the Enhanced Efficiency of Immunostimulatory Nucleic Acids,” Innate Immun. 19(1):53-65 (2013), which is hereby incorporated by reference in its entirety). Suitable TLR-4 agonists include, without limitation, MPL (Engel et al., “The Pharmacokinetics of Toll-like Receptor Agonists and the Impact on the Immune System,” Expert Rev. Clin. Pharmacol. 4(2):275-289 (2011), which is hereby incorporated by reference in its entirety), Glucopyranosyl Lipid-A (Matzner et al., “Perioperative treatment with the new synthetic TLR-4 agonist GLA-SE reduces cancer metastasis without adverse effects,” Int. J Cancer 138(7):1754-64 (2016), which is hereby incorporated by reference in its entirety), and Immunomax® (Ghochikyan et al., “Targeting TLR-4 with a novel pharmaceutical grade plant derived agonist, Immunomax®, as a therapeutic strategy for metastatic breast cancer,” J. Trans. Med. 12:322 (2014), which is hereby incorporated by reference in its entirety)

In any embodiment, the macrophage type-1 stimulating agent is a TLR-7 agonist. Suitable TLR-7 agonists include, without limitation, uridine/guanidine-rich single-stranded RNA (Engel et al., “The Pharmacokinetics of Toll-like Receptor Agonists and the Impact on the Immune System,” Expert Rev. Clin. Pharmacol. 4(2):275-289 (2011), which is hereby incorporated by reference in its entirety), 852A (Dudek et al., “First in Human Phase I Trial of 852A, a Novel Systemic Toll-like Receptor 7 Agonist, to Activate Innate Immune Responses in Patients With Advanced Cancer,” Clin. Cancer Res. 13(23):7119-7125 (2007), which is hereby incorporated by reference in its entirety), resiquimod (Chang et al., “Topical resiquimod Promotes Priming of CTL to Parenteral Antigens,” Vaccine 27(42):5791-5799 (2009), which is hereby incorporated by reference in its entirety), imidazoquinolines (Itoh et al., “The Clathrin-mediated Endocytic Pathway Participates in dsRNA-induced IFN-beta Production,” J. Immunol. 181:5522-9 (2008), which is hereby incorporated by reference in its entirety), ANA975 (Fletcher et al., “Masked oral Prodrugs of Toll-like Receptor 7 Agonists: a New Approach for the Treatment of Infectious Disease,” Curr. Opin. Investig. Drugs 7(8):702-708 (2006), which is hereby incorporated by reference in its entirety), and imiquimod (Engel et al., “The Pharmacokinetics of Toll-like Receptor Agonists and the Impact on the Immune System,” Expert Rev. Clin. Pharmacol. 4(2):275-289 (2011), which is hereby incorporated by reference in its entirety).

In any embodiment, the macrophage type-1 stimulating agent is a TLR-8 agonist. Suitable TLR-8 agonists include, without limitation, resiquimod (Chang et al., “Topical resiquimod Promotes Priming of CTL to Parenteral Antigens,” Vaccine 27(42):5791-5799 (2009), which is hereby incorporated by reference in its entirety), and imidazoquinolines (Itoh et al., “The Clathrin-mediated Endocytic Pathway Participates in dsRNA-induced IFN-beta Production,” J Immunol. 181:5522-9 (2008), which is hereby incorporated by reference in its entirety).

In any embodiment, the macrophage type-1 stimulating agent is a TLR-9 agonist. Suitable TLR-9 agonists include, without limitation, CpG-ODN (Yao et al., “Late Endosome/Lysosome-localized Rab7b Suppresses TLR-9-initiated Proinflammatory Cytokine and Type I IFN Production in Macrophages,” J Immunol. 183:1751-8 (2009), which is hereby incorporated by reference in its entirety). Specific CpG-ODNs suitable for use are described in Engel et al., “The Pharmacokinetics of Toll-like Receptor Agonists and the Impact on the Immune System,” Expert Rev. Clin. Pharmacol. 4(2):275-289 (2011), which is hereby incorporated by reference in its entirety.

Other agents known in the art to reprogram type-2 macrophages to type-1 macrophages (i.e., macrophage type-1 stimulating agent) for purposes of inclusion in the NPC1 binding peptide conjugate described herein include, manganese dioxide nanoparticles (see e.g., Song et al., “Bioconjugated Manganese Dioxide Nanoparticles Enhance Chemotherapy Response by Priming Tumor-Associated Macrophages toward MI-like Phenotype and Attenuating Tumor Hypoxia” ACSNano. 10:633-647 (2016), which is hereby incorporated by reference in its entirety), ferumoxytal nanoparticles (Zanganeh, et al. “Iron oxide nanoparticles inhibit tumour growth by inducing pro-inflammatory macrophage polarization in tumour tissues,” Nat. Nanotechnol. 11:986-994 (2016), which is hereby incorporated by reference in its entirety), mannosylated nanoparticles encapsulating siRNA against IκBα (Ortega et al. “Manipulating the NF-kappaB pathway in macrophages using mannosylated, siRNA-delivering nanoparticles can induce immunostimulatory and tumor cytotoxic functions,” Int. J. Nanomed. 2163-2177 (2016), which is hereby incorporated by reference in its entirety).

In some embodiments, the pharmaceutically active moiety of the NPC1 binding peptide conjugate is a macrophage type-2 stimulating agent. Suitable macrophage type-2 stimulating agents include, without limitation, IL-33, IL-4 receptor agonists, glucocorticoids, IL-10 receptor agonist, IL-1 receptor agonist, and analogs and derivatives thereof.

In any embodiment, the macrophage type-2 stimulating agent is an IL-4 receptor agonist. Suitable IL-4 receptor agonists include, without limitation, mutant IL-4 proteins. Exemplary mutant IL-4 proteins include, but are not limited to those described in U.S. Pat. No. 5,723,118 to Sebald, which is hereby incorporated by reference in its entirety.

In any embodiment, the macrophage type-2 stimulating agent is a glucocorticoid. Glucocorticoids are a class of corticosteroids, which are well known in the art and suitable for inducing a macrophage type-2 phenotype. Exemplary glucocorticoids for incorporation into the NPC1 binding peptide conjugate of the present disclosure include, without limitation, cortisol, cortisone, prednisone, prednisolone, methylprednisolone, dexamethasone, betamethasone, triamcinolone, beclomethasone, fludrocortisone, deoxycorticosterone, and aldosterone.

In any embodiment, the macrophage type-2 stimulating agent is an IL-10 receptor agonist. Suitable IL-10 receptor agonists include, without limitation, mutant IL-10 proteins as described in U.S. Pat. No. 7,749,490 to Sommer et al., which is hereby incorporated by reference in its entirety.

In any embodiment, the macrophage type-2 stimulating agent is an IL-1 receptor agonist. Suitable IL-1 receptor agonists include, without limitation, IL-1α, IL-1β, IL-18, IL-33, IL-36a, IL-360, and IL-367 (Palomo et al., “The Interleukin (IL)-1 Cytokine Family-Balance Between Agonists and Antagonists in Inflammatory Diseases,” Cytokine 76(1):25-37 (2015), which is hereby incorporated by reference in its entirety).

In any embodiment, the pharmaceutically active moiety of the NPC1 binding peptide conjugate is a T cell stimulating agent. In any embodiment, the T cell stimulating agent is a stimulator of interferon genes (STING) agonist. Suitable STING agonists include, without limitation, cyclic dinucleotides (CDNs), such as cyclic dimeric guanosine monophosphate (c-di-GMP), cyclic dimeric adenosine monophosphate (c-di-AMP), cyclic GMP-AMP (cGAMP), and dithio-(R_{P},R_{P})-[cyclic[A(2′,5′)pA(3′,5′)p (ADU-S100, Aduro Biotech) and small molecules, such as 5,6-dimethylxanthenone-4-acetic acid (DMXAA) and linked amidobenzimidazole. Other STING agonists under development that are also suitable immunomodulatory agents in accordance with the present disclosure include BMS-986301, E7766, GSK3745417, MK-1454, MK-2118, and SB11285.

In any embodiment, the pharmaceutically active moiety of the NPC1 binding peptide conjugate is a dendritic cell stimulating agent. Suitable dendritic cell stimulating agents include, without limitations, CpG oligonucleotides, imiquimod, topoisomerase I inhibitors (e.g. camptothecin and derivatives thereof), microtubule depolymerizing drugs (e.g. colchicine, podophyllotoxin, and derivatives thereof), and analogs and derivatives thereof.

In any embodiment, the pharmaceutically active moiety of the NPC1 binding peptide conjugate is a neutrophil stimulating agent. Suitable neutrophil stimulating agents include recombinant granulocyte colony stimulating factor proteins (filgrastim) and pegylated recombinant granulocyte colony stimulating factor proteins.

In some embodiments, the pharmaceutically active moiety of the NPC1 binding peptide conjugate is a nucleic acid molecule. Suitable nucleic acid molecule active moieties include, without limitation, an antisense oligonucleotide, an siRNA, an aptamer, an miRNA, an immunostimulatory oligonucleotide, a splice-switching oligonucleotide, and guide RNA, and analogs and derivatives thereof.

In any embodiment, the pharmaceutically active moiety of the NPC1 binding peptide conjugate is coupled to or packaged within a delivery vehicle. Accordingly, in some embodiments, the NPC1 binding peptide conjugate comprises the NPC1 binding polypeptide coupled to a delivery vehicle. In any embodiment, the delivery vehicle contains a pharmaceutically active moiety.

In accordance with this aspect of the disclosure, any suitable drug delivery vehicle known in the art can be coupled to the NPC1 binding polypeptide to form the NPC1 binding peptide conjugate as described herein. In any embodiment, the drug delivery vehicle is a nanoparticle delivery vehicle, a polymer-based particle, or a lipid-based particle delivery vehicle known in the art (see, e.g., Xiao et al., “Engineering Nanoparticles for Targeted Delivery of Nucleic Acid Therapeutics in Tumor,” Mol. Ther. Meth. Clin. Dev. 12: 1-18 (2019) and Ni et al., “Synthetic Approaches for Nucleic Acid Delivery: Choosing the Right Carriers,” Life 9(3): 59 (2019), which are hereby incorporated by reference in their entirety), can be employed in the methods as described herein.

Suitable nanoparticle delivery vehicles comprise, without limitation, gold nanoparticles, calcium phosphate nanoparticles, cadinum (quantum dots) nanoparticles, iron oxide nanoparticles, as well as particles derived from any other solid inorganic materials as known in the art.

Suitable polymer-based particles or polyplex carriers comprise cationic polymers such as polyethylenimine (PEI), and/or cationic polymers conjugated to neutral polymers, like polyethylene glycol (PEG) and cyclodextrin. Other suitable PEI conjugates to facilitate nucleic acid molecule or expression vector delivery in accordance with the methods described herein include, without limitation, PEI-salicylamide conjugates and PEI-steric acid conjugate. Other synthetic cationic polymers suitable for use as a delivery vehicle material include, without limitation, poly-L-lysine (PLL), polyacrylic acid (PAA), polyamideamine-epichlorohydrin (PAE) and poly[2-(dimethylamino)ethyl methacrylate] (PDMAEMA). Natural cationic polymers suitable for use as delivery vehicle material include, without limitation, chitosan, poly(lactic-co-glycolic acid) (PLGA), gelatin, dextran, cellulose, and cyclodextrin.

Suitable lipid-based vehicles include cationic lipid based lipoplexes (e.g., 1,2-dioleoyl-3trimethylammonium-propane (DOTAP)), neutral lipids based lipoplexes (e.g., cholesterol and dioleoylphosphatidyl ethanolamine (DOPE)), anionic lipid based lipoplexes (e.g., cholesteryl hemisuccinate (CHEMS)), and pH-sensitive lipid lipoplexes (e.g., 2,3-dioleyloxy-N-[2(sperminecarboxamido)ethyl]-N,N-dimethyl-1-propanaminium trifluoroacetate (DOSPA)). Other suitable lipid-based delivery particles incorporate ionizable DOSPA in lipofectamine and DLin-MC3-DMA ((6Z,9Z,28Z,31Z)-heptatriaconta-6,9,28,31-tetraen-19-yl-4-(dimethylamino) butanoate).

In some embodiments, the cancer therapeutic is a PROTAC. Suitable PROTACs include, without limitation BET degraders, such as that disclosed by Pillow et al., “Antibody Conjugation of a Chimeric BET Degrader Enables In vivo Activity,” ChemMedChem 15(1): 17-25 (2020), which is hereby incorporated by reference in its entirety. Suitable PROTACs also include Ras pathway degraders, see e.g., Ras pathway degraders described by Bond et al., “Targeted Degradation of Oncogenic KRAS (G12C) by VHL-recruiting PROTACs,” ACS Cent. Sci. 6(8):1367-75 (2020); Nabet et al., “The dTAG system for immediate and target-specific protein degradation,” Nat Chem Biol. 14(5):431-41 (2018); Simpson et al., “Inducible degradation of target proteins through a tractable affinity-directed protein missile system,” Cell Chem Biol. 27(9):1164-80.e5 (2020); Cheng et al., “Discovery of novel PDES degraders for the treatment of KRAS mutant colorectal cancer,” J Med Chem. 63(14):7892-905 (2020); Crew et al., “Identification and Characterization of Von Hippel-Lindau-recruiting proteolysis targeting chimeras (PROTACs) of TANK-binding kinase 1,” J Med Chem. 61(2):583-98 (2018); Vollmer et al., “Design, Synthesis, and Biological Evaluation of MEK PROTACs,” J Med Chem. 63(1):157-62 (2020); and Yang et al., “Discovery of thalidomide-based PROTAC small molecules as the highly efficient SHP2 degraders,” Eur J Med Chem. 218:113341 (2021), which are hereby incorporated by reference in their entirety.

In another embodiment, the second portion of the NPC1 binding peptide conjugate comprises a second polypeptide. In some embodiments, the second polypeptide is a non-binding molecule. In some embodiments, the polypeptide is a second binding molecule. In some embodiments, the second binding molecule is an antibody or antibody binding domain thereof. As used herein, an antibody includes any protein or peptide containing molecule that comprises at least a portion of an immunoglobulin molecule, such as but not limited to, at least one, at least two, or at least three complementarity determining region (CDR) of a heavy or light chain, a heavy chain or light chain variable region, a heavy chain or light chain constant region, a framework region, or any portion thereof. Antibodies encompass full antibodies, digestion fragments, specified portions and variants thereof, including, without limitation, portions of antibodies that mimic the structure and/or function of an antibody or specified fragment or portion thereof, including, without limitation, single chain antibodies, single domain antibodies (i.e., antibody fragments comprising merely one variable domain, which might be VHH, VH or VL, that specifically bind an antigen or epitope independently of other V regions or domains). Functional fragments include antigen-binding fragments that bind to a particular target. For example, antibody fragments capable of binding to a particular target or portions thereof, include, but are not limited to, Fab (e.g., by papain digestion), Fab′ (e.g., by pepsin digestion and partial reduction) and F(ab′)₂ (e.g., by pepsin digestion), Fd (e.g., by pepsin digestion, partial reduction and reaggregation), Fv or scFv (e.g., by molecular biology techniques) fragments.

Another aspect of the present disclosure is directed to polynucleotides encoding the NPC1 binding molecules or the NPC1 binding peptide conjugates described herein. The nucleic acid molecules of the present disclosure include isolated polynucleotides, portions of expression vectors or portions of linear DNA sequences, including linear DNA sequences used for in vitro transcription/translation, vectors compatible with prokaryotic, eukaryotic or filamentous phage expression, secretion and/or display of the compositions or directed mutagens thereof.

In one embodiment isolated polynucleotides of the present disclosure include those encoding the binding molecules described supra. Exemplary isolated polynucleotide molecules include those encoding a FN3 domain comprising a modified FG loop amino acid sequence of SEQ ID NO: 2, a modified BC loop amino acid sequence of SEQ ID NO: 15, and a modified DE loop amino acid sequence of SEQ ID NO: 30. In some embodiments, the FN domain encoded by the polynucleotide further comprises an amino acid substitution at one or more residues corresponding to residues D3, R6, D7, Y31, R33, E47, T49, and A74 of SEQ ID NO: 1. In some embodiments, the FN domain encoded by the polynucleotide of the disclosure comprises amino acid substitutions at the residues corresponding to residue D3, R6, and D7. In some embodiments, the FN3 domain encoded by the polynucleotide of the disclosure comprises an amino acid sequence that is at least 80% identical to an amino acid sequence of SEQ ID NO: 32. In some embodiments, the FN3 domain encoded by the polynucleotide of the present disclosure comprises an amino acid sequence that is at least 90% identical to an amino acid sequence of SEQ ID NO: 32. In some embodiments, the polynucleotide of the present disclosure encodes an FN3 domain comprising an amino acid sequence of SEQ ID NO: 32 (MbNPClN-N8).

In some embodiments, the isolated polynucleotide of the present disclosure encodes an NCP1 binding polypeptide having an FN3 domain comprising a modified FG loop amino acid sequence of SEQ ID NO: 3, a modified BC loop amino acid sequence of SEQ ID NO: 16, and a modified DE loop amino acid sequence of SEQ ID NO: 30. In some embodiments, the FN domain encoded by the polynucleotide of the present disclosure further comprises an amino acid substitution at one or more residues corresponding to residues D3, R6, D7, Y31, R33, E47, T49, and A74 of SEQ ID NO: 1. In some embodiments, the FN domain encoded by the polynucleotide of the disclosure comprises amino acid substitutions at the residues corresponding to residue D3, R6, and D7. In some embodiments, the FN3 domain encoded by the polynucleotide of the present disclosure comprises an amino acid sequence that is at least 80% identical to an amino acid sequence of SEQ ID NO: 33. In some embodiments, the FN3 domain comprises an amino acid sequence that is at least 90% identical to an amino acid sequence of SEQ ID NO: 33. In some embodiments, the polynucleotide of the present disclosure encodes an FN3 domain comprising an amino acid sequence of SEQ ID NO: 33 (MbNPClN-N16).

In some embodiments, the isolated polynucleotide of the present disclosure encodes an NCP1 binding polypeptide having an FN3 domain comprising a modified FG loop amino acid sequence of SEQ ID NO: 4, a modified BC loop amino acid sequence of SEQ ID NO: 17, and a modified DE loop amino acid sequence of SEQ ID NO: 30. In some embodiments, the FN domain encoded by the polynucleotide of the present disclosure further comprises an amino acid substitution at one or more residues corresponding to residues D3, R6, D7, Y31, R33, E47, T49, and A74 of SEQ ID NO: 1. In some embodiments, the FN domain encoded by the polynucleotide of the disclosure comprises amino acid substitutions at the residues corresponding to residue D3, R6, and D7. In some embodiments, the FN3 domain encoded by the polynucleotide of the present disclosure comprises an amino acid sequence that is at least 80% identical to an amino acid sequence of SEQ ID NO: 34. In some embodiments, the FN3 domain comprises an amino acid sequence that is at least 90% identical to an amino acid sequence of SEQ ID NO: 34. In some embodiments, the polynucleotide of the present disclosure encodes an FN3 domain comprising an amino acid sequence of SEQ ID NO: 34 (MbNPClN-N18).

In some embodiments, the isolated polynucleotide of the present disclosure encodes an NCP1 binding polypeptide having an FN3 domain comprising a modified FG loop amino acid sequence of SEQ ID NO: 5, a modified BC loop amino acid sequence of SEQ ID NO: 18, and a modified CD loop amino acid sequence of SEQ ID NO: 23. In some embodiments, the FN domain encoded by the polynucleotide of the present disclosure further comprises an amino acid substitution at one or more residues corresponding to residues D3, R6, D7, Y31, R33, E47, T49, and A74 of SEQ ID NO: 1. In some embodiments, the FN domain encoded by the polynucleotide of the disclosure comprises amino acid substitutions at the residues corresponding to residue D3, R6, D7, R33, and E47. In some embodiments, the FN3 domain encoded by the polynucleotide of the present disclosure comprises an amino acid sequence that is at least 80% identical to an amino acid sequence of SEQ ID NO: 35. In some embodiments, the FN3 domain comprises an amino acid sequence that is at least 90% identical to an amino acid sequence of SEQ ID NO: 35. In some embodiments, the polynucleotide of the present disclosure encodes an FN3 domain comprising an amino acid sequence of SEQ ID NO: 35 (MbNPC1N-N22).

In some embodiments, the isolated polynucleotide of the present disclosure encodes an NCP1 binding polypeptide having an FN3 domain comprising a modified FG loop amino acid sequence of SEQ ID NO: 6, a modified BC loop amino acid sequence of SEQ ID NO: 19, and a modified CD loop amino acid sequence of SEQ ID NO: 23. In some embodiments, the FN domain encoded by the polynucleotide of the present disclosure further comprises an amino acid substitution at one or more residues corresponding to residues D3, R6, D7, Y31, R33, E47, T49, and A74 of SEQ ID NO: 1. In some embodiments, the FN domain encoded by the polynucleotide of the disclosure comprises amino acid substitutions at the residues corresponding to residue D3, R6, D7, E47, and A74. In some embodiments, the FN3 domain encoded by the polynucleotide of the present disclosure comprises an amino acid sequence that is at least 80% identical to an amino acid sequence of SEQ ID NO: 36. In some embodiments, the FN3 domain comprises an amino acid sequence that is at least 90% identical to an amino acid sequence of SEQ ID NO: 36. In some embodiments, the polynucleotide of the present disclosure encodes an FN3 domain comprising an amino acid sequence of SEQ ID NO: 36 (MbNPC1N-N23).

In some embodiments, the isolated polynucleotide of the present disclosure encodes an NCP1 binding polypeptide having an FN3 domain comprising a modified FG loop amino acid sequence of SEQ ID NO: 7, a modified BC loop amino acid sequence of SEQ ID NO: 18, and a modified CD loop amino acid sequence of SEQ ID NO: 24. In some embodiments, the FN domain encoded by the polynucleotide of the present disclosure further comprises an amino acid substitution at one or more residues corresponding to residues D3, R6, D7, Y31, R33, E47, T49, and A74 of SEQ ID NO: 1. In some embodiments, the FN domain encoded by the polynucleotide of the disclosure comprises amino acid substitutions at the residues corresponding to residue D3, R6, D7, R33, E47, and T49. In some embodiments, the FN3 domain encoded by the polynucleotide of the present disclosure comprises an amino acid sequence that is at least 80% identical to an amino acid sequence of SEQ ID NO: 37. In some embodiments, the FN3 domain comprises an amino acid sequence that is at least 90% identical to an amino acid sequence of SEQ ID NO: 37. In some embodiments, the polynucleotide of the present disclosure encodes an FN3 domain comprising an amino acid sequence of SEQ ID NO: 37 (MbNPC1N-N24).

In some embodiments, the isolated polynucleotide of the present disclosure encodes an NCP1 binding polypeptide having an FN3 domain comprising a modified FG loop amino acid sequence of SEQ ID NO: 8, a modified BC loop amino acid sequence of SEQ ID NO: 18, and a modified CD loop amino acid sequence of SEQ ID NO: 25. In some embodiments, the FN domain encoded by the polynucleotide of the present disclosure further comprises an amino acid substitution at one or more residues corresponding to residues D3, R6, D7, Y31, R33, E47, T49, and A74 of SEQ ID NO: 1. In some embodiments, the FN domain encoded by the polynucleotide of the disclosure comprises amino acid substitutions at the residues corresponding to residue D3, R6, D7, R33, and E47. In some embodiments, the FN3 domain encoded by the polynucleotide of the present disclosure comprises an amino acid sequence that is at least 80% identical to an amino acid sequence of SEQ ID NO: 38. In some embodiments, the FN3 domain comprises an amino acid sequence that is at least 90% identical to an amino acid sequence of SEQ ID NO: 38. In some embodiments, the polynucleotide of the present disclosure encodes an FN3 domain comprising an amino acid sequence of SEQ ID NO: 38 (MbNPC1N-N26).

In some embodiments, the isolated polynucleotide of the present disclosure encodes an NCP1 binding polypeptide having an FN3 domain comprising a modified FG loop amino acid sequence of SEQ ID NO: 9, a modified BC loop amino acid sequence of SEQ ID NO: 18, and a modified CD loop amino acid sequence of SEQ ID NO: 26. In some embodiments, the FN domain encoded by the polynucleotide of the present disclosure further comprises an amino acid substitution at one or more residues corresponding to residues D3, R6, D7, Y31, R33, E47, T49, and A74 of SEQ ID NO: 1. In some embodiments, the FN domain encoded by the polynucleotide of the disclosure comprises amino acid substitutions at the residues corresponding to residue D3, R6, D7, R33, and E47. In some embodiments, the FN3 domain encoded by the polynucleotide of the present disclosure comprises an amino acid sequence that is at least 80% identical to an amino acid sequence of SEQ ID NO: 39. In some embodiments, the FN3 domain comprises an amino acid sequence that is at least 90% identical to an amino acid sequence of SEQ ID NO: 39. In some embodiments, the polynucleotide of the present disclosure encodes an FN3 domain comprising an amino acid sequence of SEQ ID NO: 39 (MbNPC1N-N31).

In some embodiments, the isolated polynucleotide of the present disclosure encodes an NCP1 binding polypeptide having an FN3 domain comprising a modified FG loop amino acid sequence of SEQ ID NO: 10, a modified BC loop amino acid sequence of SEQ ID NO: 18, and a modified CD loop amino acid sequence of SEQ ID NO: 26. In some embodiments, the FN domain encoded by the polynucleotide of the present disclosure further comprises an amino acid substitution at one or more residues corresponding to residues D3, R6, D7, Y31, R33, E47, T49, and A74 of SEQ ID NO: 1. In some embodiments, the FN domain encoded by the polynucleotide of the disclosure comprises amino acid substitutions at the residues corresponding to residue D3, R6, D7, R33, E47, and T49. In some embodiments, the FN3 domain encoded by the polynucleotide of the present disclosure comprises an amino acid sequence that is at least 80% identical to an amino acid sequence of SEQ ID NO: 40. In some embodiments, the FN3 domain comprises an amino acid sequence that is at least 90% identical to an amino acid sequence of SEQ ID NO: 40. In some embodiments, the polynucleotide of the present disclosure encodes an FN3 domain comprising an amino acid sequence of SEQ ID NO: 40 (MbNPC1N-N34).

In some embodiments, the isolated polynucleotide of the present disclosure encodes an NCP1 binding polypeptide having an FN3 domain comprising a modified FG loop amino acid sequence of SEQ ID NO: 11, a modified BC loop amino acid sequence of SEQ ID NO: 20, and a modified CD loop amino acid sequence of SEQ ID NO: 24. In some embodiments, the FN domain encoded by the polynucleotide of the present disclosure further comprises an amino acid substitution at one or more residues corresponding to residues D3, R6, D7, Y31, R33, E47, T49, and A74 of SEQ ID NO: 1. In some embodiments, the FN domain encoded by the polynucleotide of the disclosure comprises amino acid substitutions at the residues corresponding to residue D3, R6, D7, R33, E47, and T49. In some embodiments, the FN3 domain encoded by the polynucleotide of the present disclosure comprises an amino acid sequence that is at least 80% identical to an amino acid sequence of SEQ ID NO: 41. In some embodiments, the FN3 domain comprises an amino acid sequence that is at least 90% identical to an amino acid sequence of SEQ ID NO: 41. In some embodiments, the polynucleotide of the present disclosure encodes an FN3 domain comprising an amino acid sequence of SEQ ID NO: 41 (MbNPC1N-N35).

In some embodiments, the isolated polynucleotide of the present disclosure encodes an NCP1 binding polypeptide having an FN3 domain comprising a modified FG loop amino acid sequence of SEQ ID NO: 12, a modified BC loop amino acid sequence of SEQ ID NO: 21, and a modified CD loop amino acid sequence of SEQ ID NO: 27. In some embodiments, the FN domain encoded by the polynucleotide of the present disclosure further comprises an amino acid substitution at one or more residues corresponding to residues D3, R6, D7, Y31, R33, E47, T49, and A74 of SEQ ID NO: 1. In some embodiments, the FN domain encoded by the polynucleotide of the disclosure comprises amino acid substitutions at the residues corresponding to residue D3, R6, D7, Y31, R33, and E47. In some embodiments, the FN3 domain encoded by the polynucleotide of the present disclosure comprises an amino acid sequence that is at least 80% identical to an amino acid sequence of SEQ ID NO: 42. In some embodiments, the FN3 domain comprises an amino acid sequence that is at least 90% identical to an amino acid sequence of SEQ ID NO: 42. In some embodiments, the polynucleotide of the present disclosure encodes an FN3 domain comprising an amino acid sequence of SEQ ID NO: 42 (MbNPC1N-N38).

In some embodiments, the isolated polynucleotide of the present disclosure encodes an NCP1 binding polypeptide having an FN3 domain comprising a modified FG loop amino acid sequence of SEQ ID NO: 13, a modified BC loop amino acid sequence of SEQ ID NO: 20, and a modified CD loop amino acid sequence of SEQ ID NO: 28. In some embodiments, the FN domain encoded by the polynucleotide of the present disclosure further comprises an amino acid substitution at one or more residues corresponding to residues D3, R6, D7, Y31, R33, E47, T49, and A74 of SEQ ID NO: 1. In some embodiments, the FN domain encoded by the polynucleotide of the disclosure comprises amino acid substitutions at the residues corresponding to residue D3, R6, D7, Y31, R33, E47, and T49. In some embodiments, the FN3 domain encoded by the polynucleotide of the present disclosure comprises an amino acid sequence that is at least 80% identical to an amino acid sequence of SEQ ID NO: 43. In some embodiments, the FN3 domain comprises an amino acid sequence that is at least 90% identical to an amino acid sequence of SEQ ID NO: 43. In some embodiments, the polynucleotide of the present disclosure encodes an FN3 domain comprising an amino acid sequence of SEQ ID NO: 43 (MbNPC1C-C45).

The polynucleotides of the disclosure may be produced by chemical synthesis such as solid phase polynucleotide synthesis on an automated polynucleotide synthesizer and assembled into complete single or double stranded molecules. Alternatively, the polynucleotides of the disclosure may be produced by other techniques such PCR followed by routine cloning. Techniques for producing or obtaining polynucleotides of a given known sequence are well known in the art.

The polynucleotides described herein may comprise at least one non-coding sequence, such as a promoter or enhancer sequence, intron, polyadenylation signal, a cis sequence facilitating RepA binding, and the like. The polynucleotide sequences may also comprise additional sequences encoding additional amino acids that encode for example a marker or a tag sequence such as a histidine tag or an HA tag to facilitate purification or detection of the protein, a signal sequence, a fusion protein partner such as RepA, Fc or bacteriophage coat protein such as pIX or pIII.

Another embodiment of the disclosure is a vector comprising at least one or more of the polynucleotides as described herein. Such vectors may be plasmid vectors, viral vectors, vectors for baculovirus expression, transposon based vectors or any other vector suitable for introduction of the polynucleotides of the invention into a given organism or genetic background by any means. Such vectors may be expression vectors comprising nucleic acid sequence elements that can control, regulate, cause or permit expression of a polypeptide encoded by such a vector. Such elements may comprise transcriptional enhancer binding sites, RNA polymerase initiation sites, ribosome binding sites, and other sites that facilitate the expression of encoded polypeptides in a given expression system. Such expression systems may be cell-based, or cell-free systems well known in the art.

Another embodiment of the present disclosure is a host cell comprising the above described vectors. The binding molecules and/or NPC1 binding peptide conjugates disclosed herein can be optionally produced by a cell line, a mixed cell line, an immortalized cell or clonal population of immortalized cells, as well known in the art (see e.g., Ausubel et al., ed., Current Protocols in Molecular Biology, John Wiley & Sons, Inc., NY, N.Y. (1987-2001); Sambrook et al., Molecular Cloning: A Laboratory Manual, ₂^{nd} Edition, Cold Spring Harbor, N.Y. (1989); Harlow and Lane, Antibodies, a Laboratory Manual, Cold Spring Harbor, N.Y. (1989); Colligan et al., eds., Current Protocols in Immunology, John Wiley & Sons, Inc., NY (1994-2001); Colligan et al., Current Protocols in Protein Science, John Wiley & Sons, NY, N.Y., (1997-2001), which are hereby incorporated by reference in their entirety).

The host cell chosen for expression may be of mammalian origin or may be selected from COS-1, COS-7, HEK293, BHK21, CHO, BSC-1, He G2, SP2/0, HeLa, myeloma, lymphoma, yeast, insect or plant cells, or any derivative, immortalized or transformed cell thereof. Alternatively, the host cell may be selected from a species or organism incapable of glycosylating polypeptides, e.g. a prokaryotic cell or organism, such as BL21, BL21(DE3), BL21-GOLD(DE3), XL1-Blue, JM109, HMS174, HMS174(DE3), and any of the natural or 30 engineered E. coli spp, Klebsiellaspp., or Pseudomonas spp strains.

Another aspect of the disclosure is directed to a method of producing and isolating the binding molecules and NPC1 binding peptide conjugates as described herein. This method involves culturing the isolated host cell of the disclosure under conditions such that the binding molecules or NPC1 binding peptide conjugates are expressed, and purifying the expressed binding molecules or NPC1 binding peptide conjugates from the host cell culture.

The binding molecules and NPC1 binding peptide conjugates described herein can be purified from recombinant cell cultures by well-known methods, for example by protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography, or high performance liquid chromatography (HPLC).

Purified or isolated binding molecules and NPC1 binding peptide conjugates as described herein may be linked to one of a variety of non-proteinaceous polymers, e.g., polyethylene glycol, polypropylene glycol, polyoxyalkylenes, or copolymers of polyethylene glycol and polypropylene glycol. The binding molecules and/or NPC1 binding peptide conjugates may also be entrapped in microcapsules prepared, for example, by coacervation techniques or by interfacial polymerization (for example, hydroxymethylcellulose or gelatine-microcapsules and poly (methylmethacylate) microcapsules, respectively), in colloidal drug delivery systems (for example, liposomes, albumin microspheres, microemulsions, nanoparticles and nanocapsules), or in macroemulsions. Such techniques are disclosed in REMINGTON'S PHARMACEUTICAL SCIENCES, 16th edition, Oslo, A., Ed., (1980), which is hereby incorporated by reference in its entirety.

For therapeutic use, the binding molecules and NPC1 binding peptide conjugates as described herein may be prepared as pharmaceutical compositions containing an effective amount of the binding molecule or NPC1 binding peptide conjugate as an active ingredient in a pharmaceutically acceptable carrier. The term “carrier” refers to a diluent, adjuvant, excipient, or vehicle with which the active compound is administered. Such vehicles can be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of binding molecule or NPC1 binding peptide conjugate as described herein in such pharmaceutical formulation can vary widely, i.e., from less than about 0.5%, usually at or at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. REMINGTON: THE SCIENCE AND PRACTICE OF PHARMACY, 21^{st} Edition, Troy, D. B. ed., Lipincott Williams and Wilkins, 2006, Part 5, Pharmaceutical Manufacturing pp 691-1092, see especially pp. 958-989, which is hereby incorporated by reference in its entirety.

The binding molecules and NPC1 binding peptide conjugates described herein can be used in non-isolated or isolated form. Furthermore, the binding molecules and NPC1 binding peptide conjugates hereof can be used alone or in a mixture comprising at least one other binding molecule or NPC1 binding peptide conjugate hereof. In other words, the binding molecules and NPC1 binding peptide conjugates can be used in combination, e.g., as a pharmaceutical composition comprising two or more binding molecules hereof, two or more NPC1 binding peptide conjugates, a binding molecule and NPC1 binding peptide conjugate, and variants thereof. For example, binding molecules and/or NPC1 binding peptide conjugates having different, but complementary activities can be combined in a single therapy to achieve a desired therapeutic effect, but alternatively, binding molecules and NPC1 binding peptide conjugates having identical activities can also be combined in a single therapy to achieve a desired therapeutic or diagnostic effect. Optionally, the mixture further comprises at least one other therapeutic agent.

Another aspect of the present disclosure relates to a combination therapeutic. This combination therapeutic includes the NPC1 binding polypeptide as described herein and a pharmaceutically active moiety.

In accordance with this aspect of the disclosure, the pharmaceutically active moiety of the combination therapeutic can be any pharmaceutically active moiety known in the art. Suitable pharmaceutically active moieties include, without limitation, small molecule active moieties, nucleic acid molecules, antibodies, antibody binding fragments, antibody derivatives, a protein or polypeptide fragment thereof, a proteolysis targeting chimera (PROTAC), and analogs and derivatives thereof.

As used herein, the term “combination therapy” refers to the administration of two or more therapeutic agents, i.e., the NPC1 binding polypeptide or NPC1 binding peptide conjugate comprising the same as described herein, in combination with an active pharmaceutical moiety. In some embodiments, the combination therapy is co-administered in a substantially simultaneous manner, such as in a single capsule or other delivery vehicle having a fixed ratio of active ingredients. In some embodiments, the combination therapy is administered in multiple capsules or delivery vehicles, each containing an active ingredient. In some embodiments, the therapeutic agents of the combination therapy are administered in a sequential manner, either at approximately the same time or at different times. For example, in one embodiment, the NPC1 binding polypeptide as described herein is administered as a neo-adjuvant, i.e., it is administered prior to the administration of the pharmaceutically active moiety. In other embodiments, the NPC1 binding polypeptide is administered as a standard adjuvant therapy, i.e., it is administered after the administration of the pharmaceutically active moiety. In all of the embodiments, the combination therapy provides beneficial effects of the drug combination in treating a particular condition, e.g., for treating cancer, particularly in early stage, aggressive and treatment-resistant cancers.

In any embodiment, the pharmaceutically active moiety of the combination therapeutic is a cancer therapeutic. In some embodiments, the cancer therapeutic of the combination therapeutic is a chemotherapeutic. Suitable chemotherapeutics include, without limitation, alkylating agents (e.g., chlorambucil, cyclophophamide, CCNU, melphalan, procarbazine, thiotepa, BCNU, and busulfan), antimetabolites (e.g., methotraxate, 6-mercaptopurine, and 5-fluorouracil), anthracyclines (daunorubicin, doxorubicin, idarubicin, epirubicin, and mitoxantrone), antitumor antibiotics (e.g., bleomycin, monoclonal antibodies (e.g., Alemtuzumab, Bevacizumab, Cetuximab, Gemtuzumab, Ibritumomab, Panitumumab, Rituximab, Tositumomab, and Trastuxmab), platiniums (e.g., cisplatin and oxaliplatin) or plant alkaloids (e.g., topoisomerase inhibitors, vinca alkaloids, taxanes (e.g. paclitaxel), and epipodophyllotoxins). In some embodiments, the cancer chemotherapeutic is selected from cyclophosphamide, gemcitabine, vorinostat, temozolomide, bortezomib, carmustine, and paclitaxel.

In some embodiments, the cancer therapeutic of the combination therapeutic is an immune checkpoint inhibitor. Suitable immune checkpoint inhibitors include, without limitation, a CTLA-4 inhibitor, a PD-1 inhibitor, and a PD-L1 inhibitor. In some embodiments, the immune checkpoint inhibitor is a PD-1 inhibitor selected from Pembrolizumab (Keytruda), Nivolumab (Opdivo), and Cemiplimab (Libtayo). In some embodiments, the immune checkpoint inhibitor is a PD-L1 inhibitor selected from Atezolizumab (Tecentriq), Avelumab (Bavencio), and Durvalumab (Imfinzi). In come embodiments, the immune checkpoint inhibitor is a CTLA-4 inhibitor, such as Ipilimumab (Yervoy).

In some embodiments, the cancer therapeutic of the combination therapeutic is an epidermal growth factor (EGFR) inhibitor. Suitable EGFR inhibitors include, without limitation, gefitinib, erlotinib, lapatinib, cetuximab, osimertinib, panitumumab, neratinib, vandetanib, necitumumab, and dacomitinib.

In some embodiments, the cancer therapeutic of the combination therapeutic is an mTOR inhibitor. Suitable mTOR inhibitors include, without limitation sirolimus, everolimus, temsirolimus, and everolimus.

Another aspect of the present disclosure relates to a method of treating cancer in a subject. This method involves selecting a subject having cancer and administering an NPC1 binding polypeptide as described herein, a NPC1 binding peptide conjugate comprising the NPC1 binding polypeptide as described herein, a polynucleotide encoding the NPC1 binding polypeptide or NPC1 binding peptide conjugate, or a pharmaceutical composition containing any of the aforementioned agents to the subject in an amount effective to treat the cancer.

In accordance with all of the methods described herein a “subject” refers to any animal or human having a condition that would benefit from NPC1 inhibition. In one embodiment, the subject is a mammal. Exemplary mammalian subjects include, without limitation, humans, non-human primates, dogs, cats, rodents (e.g., mouse, rat, guinea pig), horses, cattle and cows, sheep, and pigs.

In some embodiments, the subject has a type of cancer that is characterized by cancerous cells having enhanced macropinocytosis relative to their corresponding non-cancerous cells. In some embodiments, the cancer is characterized by cancerous cells having an oncogenic mutation in H-ras, N-ras, or K-ras. In some embodiments, the subject has a cancer selected from pancreatic cancer, lung cancer, breast cancer, colon cancer, glioma, solid tumor, melanoma, glioblastoma multiforme, leukemia, renal cell carcinoma, hepatocellular carcinoma, prostate cancer, and myeloma.

In some embodiments the subject has a type of cancer that is or has become resistant to primary cancer therapeutic treatment, e.g., resistant to chemotherapy treatment, prior to administering the NPC1 binding molecule or pharmaceutical composition comprising the same. Administering the NPC1 binding molecule or pharmaceutical composition comprising the same is carried out in an amount effective to re-sensitize the cancer cells to primary cancer therapeutic treatment.

In some embodiments, the method of treating a subject having cancer further involves administering a cancer therapeutic in conjunction with said NPC1 binding polypeptide, NPC1 binding peptide conjugate, or pharmaceutical composition comprising the same. Suitable cancer therapeutics that can be administered in combination with the NPC1 compositions described herein as a combination therapy are described supra.

In accordance with the methods described herein, administration of the NPC1 binding molecule or pharmaceutical composition comprising the same alone or in combination with one or more cancer therapeutics is carried out by systemic or local administration. Suitable modes of systemic administration of the therapeutic agents and/or combination therapeutics disclosed herein include, without limitation, orally, topically, transdermally, parenterally, intradermally, intrapulmonary, intramuscularly, intraperitoneally, intravenously, subcutaneously, or by intranasal instillation, by intracavitary or intravesical instillation, intraocularly, intra-arterially, intralesionally, or by application to mucous membranes. In certain embodiments, the therapeutic agents of the methods described herein are delivered orally. Suitable modes of local administration of the therapeutic agents and/or combinations disclosed herein include, without limitation, catheterization, implantation, direct injection, dermal/transdermal application, or portal vein administration to relevant tissues, or by any other local administration technique, method or procedure generally known in the art. The mode of affecting delivery of agent will vary depending on the type of therapeutic agent and the type of cancer to be treated.

A therapeutically effective amount of the NPC1 binding molecule or pharmaceutical composition comprising the same alone or in combination with a cancer therapeutic in the methods disclosed herein is an amount that, when administered over a particular time interval, results in achievement of one or more therapeutic benchmarks (e.g., slowing or halting of tumor growth, tumor regression, cessation of symptoms, etc.). The NPC1 binding molecule or pharmaceutical composition comprising the same alone or in combination with the cancer therapeutic for use in the presently disclosed methods may be administered to a subject one time or multiple times. In those embodiments where the therapeutic composition is administered multiple times, they may be administered at a set interval, e.g., daily, every other day, weekly, or monthly. Alternatively, they can be administered at an irregular interval, for example on an as-needed basis based on symptoms, patient health, and the like. For example, a therapeutically effective amount may be administered once a day (q.d.) for one day, at least 2 days, at least 3 days, at least 4 days, at least 5 days, at least 6 days, at least 7 days, at least 10 days, or at least 15 days. Optionally, the status of the cancer or the regression of the cancer is monitored during or after the treatment, for example, by a multiparametric ultrasound (mpUS), multiparametric magnetic resonance imaging (mpMRI), and nuclear imaging (positron emission tomography [PET]) of the subject. The dosage of the therapeutic agent or combination therapy administered to the subject can be increased or decreased depending on the status of the cancer or the regression of the cancer detected.

The skilled artisan can readily determine this amount, on either an individual subject basis (e.g., the amount of a compound necessary to achieve a particular therapeutic benchmark in the subject being treated) or a population basis (e.g., the amount of a compound necessary to achieve a particular therapeutic benchmark in the average subject from a given population). Ideally, the therapeutically effective amount does not exceed the maximum tolerated dosage at which 50% or more of treated subjects experience side effects that prevent further drug administrations.

A therapeutically effective amount may vary for a subject depending on a variety of factors, including variety and extent of the symptoms, sex, age, body weight, or general health of the subject, administration mode and salt or solvate type, variation in susceptibility to the drug, the specific type of the disease, and the like.

Another aspect of the present disclosure relates to a method for treating an infectious disease in a subject. This method involves selecting a subject having an infectious disease and administering the NCP1 binding polypeptide or the NPC1 binding peptide conjugate as described herein to the subject in an amount effective to treat the infectious disease.

In some embodiments, the subject having the infectious disease has a filovirus. In some embodiments, the filovirus is ebola virus or Marburg virus. Ebola and other filoviruses attach and enter a host cell via endocytosis. The internalized virus is localized in late endosomes/lysosomes and is cleaved by cysteine proteases. The cleaved Ebola glycoprotein serves as a ligand for NPC1. Inhibition of this interaction by NPC1 inhibitors block viral infection. See e.g., Basu et al., “Novel Small Molecule Entry Inhibitors of Ebola Virus,” J. Infect. Dis. 212(Suppl 2): S425-434 (2015), which is hereby incorporated by reference in its entirety. Accordingly, the NPC1 binding molecules described herein can be administered to a subject that has or is at risk of having filovirus infection as a therapeutic means of inhibiting infection, inhibiting the progression of infection, and/or decreasing infection in the subject.

In some embodiments, the subject having the infectious disease has a coronavirus. In some embodiments, the coronavirus is Severe Acute Respiratory Syndrome Coronavirus-2 (SARS-CoV-2) or Middle East Respiratory Syndrome Coronavirus (MERS-CoV). Loss of function mutations in NPC1 leads to an induction of cholesterol synthesis which has been show to combat coronavirus mediated suppression of cholesterol synthesis (Daniloski et al., “Identification of Required Host Factors for SARS-CoV-2 Infection in Human Cells,” Cell https://doi.org/10.1016/j.cell.2020.10.030 (2020), which is hereby incorporated by reference in its entirety. Accordingly, the NPC1 binding molecules described herein can be administered to a subject that has or is at risk of having a coronavirus infection as a therapeutic means of inhibiting infection, inhibiting the progression of infection, and/or decreasing infection in the subject.

Suitable pharmaceutical compositions comprising the NPC1 binding molecules and/or NPC1 binding peptide conjugates thereof for administration to a subject having an infectious disease are described supra.

Another aspect of the present disclosure is directed to a method of enhancing endosomal release of a pharmaceutically active moiety in a subject in need thereof. In one embodiment, this method involves administering, to the subject, a NPC1 binding peptide conjugate as described herein, i.e., comprising a first NPC1 binding polypeptide portion and a second portion, coupled to the first portion, where the second portion is a pharmaceutically active moiety. In another embodiment, the method involves administering, to the subject, a combination therapeutic as described herein, i.e., a combination therapeutic comprising a NPC1 binding polypeptide and a pharmaceutically active moiety.

In accordance with this aspect of the disclosure, the pharmaceutically active moiety can be any pharmaceutically active moiety known in the art, including, without limitation, a small molecule active moiety, a nucleic acid molecule active molecule, an antibody or binding fragment thereof, an antibody derivative, a protein or polypeptide fragment thereof, a proteolysis targeting chimera (PROTAC), and analogs and derivatives thereof.

In any embodiment, the subject has a neurodegenerative disease and the pharmaceutically active moiety is suitable for treating said neurodegenerative disease. Exemplary neurodegenerative diseases include, without limitation, amyotrophic lateral sclerosis, Parkinson's disease, Huntington's disease, Alzheimer's disease.

In any embodiment, the subject has amyotrophic lateral sclerosis (ALS), and the method involves administering an NPC1 binding peptide conjugate or an NPC1 combination therapeutic comprising an ALS therapeutic to treat ALS in the subject. Suitable ALS therapeutics include, without limitation, glutamate blockers (e.g. Riluzole, Rilutek, and other derivatives), Endaravone, Radicava, muscle relaxants (e.g. Baclofen, Tizanidine, and other derivatives), and analogs and derivatives thereof.

In any embodiment, the subject has Parkinson's disease, and the method involves administering an NPC1 binding peptide conjugate or an NPC1 combination therapeutic comprising a Parkinson's disease therapeutic to treat Parkinson's disease in the subject. Suitable therapeutics to treat Parkinson's disease include, without limitation, dopamine promoters (e.g., Carbidopa, Levodopa, Carbidopa-levodopa, Entacapone, Cabergoline, Tolcapone, Bromocriptine, Amantadine, and other derivatives), dopamine agonists (e.g. pramipexole, Mirapex, ropinirole, Requip, rotigotine, Neupro, apomorphine, Apokyn), cognition-enhancing medication (Rivastigmine, and other derivatives), anti-tremor drugs (e.g. Benzotropine, and other derivatives), MAO B inhibitors (selegiline, Zelapar, rasagiline, Azilect, safinamide, Xadago, and other derivatives), catechol O-methyl transferase (COMT) inhibitors (e.g. entacapone, Comtan, opicapone, Ongentys, tolcapone, Tasmar, anticholinergics (e.g. benzotropine, Cogentin, trihexyphenidyl, and other derivatives), and analogs and combinations thereof.

In any embodiment, the subject has Huntington's disease, and the method involves administering an NPC1 binding peptide conjugate or an NPC1 combination therapeutic comprising a Huntington's disease therapeutic to treat Huntington's disease in the subject. Suitable therapeutics to treat symptoms of Huntington's disease include, without limitation, movement controlling drugs (e.g. tetrabenazine, Xenazine, deutetrabenazine, Austedo, and other derivatives) antipsychotic drugs (e.g. haloperidol, Haldol, fluphenazine, risperidone, Risperdal, olanzapine, Zyprexa, quetiapine, Seroquel, and other derivatives), chorea suppressants (e.g. amantadine, Gocovri ER, Osmolex ER, levetiracetam, Keppra, Elepsia XR, Spritam, clonazepam, Klonopin, and other derivatives), and analogs and derivatives thereof.

In any embodiment, the subject has Alzheimer's disease, and the method involves administering an NPC1 binding peptide conjugate or an NPC1 combination therapeutic comprising an Alzheimer's disease therapeutic to treat Alzheimer's disease in the subject. Suitable therapeutics to treat Alzheimer's disease include, without limitation, cognition-enhancing medication (e.g. memantine, Namenda, and other derivatives), cholinesterase inhibitors (e.g. donepezil, Aricept, galantamine, Razadyne, rivastigmine, Exelon, and other derivatives), aducanumab, Aduhelm, and analogs and derivatives thereof.

In another embodiment, the method of enhancing endosomal release of a pharmaceutically active moiety involves administering the NPC1 binding peptide conjugate or NPC1 combination therapeutic to a subject having an inflammatory condition to treat the condition, where the pharmaceutically active moiety of the NCP1 binding peptide conjugate or combination therapeutic is suitable for treating said inflammatory condition. Exemplary inflammatory conditions that can be treated in accordance with this method include, without limitation, rheumatoid arthritis, atherosclerosis, macular degeneration, osteoporosis, immune inflammation, non-immune inflammation, renal inflammation, tuberculosis, multiple sclerosis, arthritis, chronic obstructive pulmonary disease (COPD), and Alzheimer's disease.

Suitable anti-inflammatory therapeutics for incorporation into the NPC1 binding peptide conjugate or NPC1 combination therapeutic include, without limitation, nonsteroidal anti-inflammatory drugs (NSAIDs) (e.g. ibuprofen, Advil, Motrin IB, naproxen sodium, Aleve, and other derivatives), corticosteroid medications (e.g. prednisone and other derivatives), conventional disease-modifying antirheumatic drugs (DMARDs) (e.g. methotrexate, Trexall, Otrexup, leflunomide, Arava, hydroxychloroquine, Plaquenil, sulfasalazine Azulfidine, and other derivatives), biologic DMARDs (abatacept, Orencia, adalimumab, Humira, anakinra, Kineret, certolizumab, Cimzia, etanercept, Enbrel, golimumab, Simponi, infliximab, Remicade, rituximab, Rituxan, sarilumab, Kevzara, tocilizumab, Actemra, and other derivatives), targeted synthetic DMARDs (e.g.baricitinib, Olumiant, tofacitinib, Xeljanz, upadacitinib, Rinvoq, and other derivatives), and analogs and derivatives thereof.

Additional anti-inflammatory therapeutics for incorporation into the NPC1 binding peptide conjugate or NPC1 combination therapeutic include, without limitation, without limitation, statins (e.g. Atorvastatin, Lovastatin, Simvastatin, Pravastatin, and other derivatives) and other cholesterol medications (e.g. exetimibe, Zetia, Fenofibrate, Gemfibrozil, and other derivatives), anticoagulants (e.g. aspirin and other derivatives), blood thinners, and analogs and derivatives thereof.

In another embodiment, the method of enhancing endosomal release of a pharmaceutically active moiety involves administering the NPC1 binding peptide conjugate or NPC1 combination therapeutic to a subject having a bone condition to treat the bone condition, where the pharmaceutically active moiety of the NCP1 binding peptide conjugate or combination therapeutic is suitable for treating said bone condition. In any embodiment, the subject has a bone conditions selected from osteoporosis or Paget's Bone disease.

In any embodiment, the subject has osteoporosis, and the method involves administering an NPC1 binding peptide conjugate or an NPC1 combination therapeutic comprising an osteoporosis therapeutic to treat the osteoporosis in the subject. Suitable osteoporosis therapeutics include, without limitation, bisphosphonates (e.g. Alendronate, Binosto, Fosamax, Ibandronate, Boniva, Risedronate, Actonel, Atelvia, Zoledronic acid, Reclast, Zometa, and other derivatives), denosumab (e.g. Prolia, Xgeva, and other derivatives), hormone-related therapy (e.g. estrogen, raloxifene, Evista, testosterone, and other derivatives), bone-building medications (e.g. Teriparatide, Bonsity, Forteo, Abaloparatide, Tymlos, Romosozumab, Evenity, and other derivatives), and analogs and derivatives thereof.

In any embodiment, the subject has Paget's bone disease, and the method involves administering an NPC1 binding peptide conjugate or an NPC1 combination therapeutic comprising a Paget's bone disease therapeutic to treat the Paget's bone disease in the subject. Suitable therapeutics for Paget's Bone disease include, without limitation, bisphosphonates (e.g. Zoledronic acid, Reclast, Zometa, Pamidronate, Aredia, Ibandronate, Boniva, and other derivatives), and oral bisphosphonates (e.g. Alendronate, Binosto, Risedronate, Actonel, Atelvia, and other derivatives), and analogs and derivatives thereof.

In another embodiment, the method of enhancing endosomal release of a pharmaceutically active moiety involves administering the NPC1 binding peptide conjugate or NPC1 combination therapeutic to a subject having cancer, where the pharmaceutically active moiety of the NCP1 binding peptide conjugate or combination therapeutic is suitable for treating the cancer. Pharmaceutically active moieties known and available to treat cancer are described in detail supra. In any embodiment, the subject has a cancer associated with RAS-pathway activation or hyperactivation (e.g., EGFR-driven cancers and PTEN deficient cancers).

In accordance with the methods described herein, administration of the NPC1 binding polypeptide, the NPC1 binding peptide conjugate, or NPC1 combination therapeutic for treatment of the various conditions described herein (e.g., cancer, infectious diseases, neurodegenerative diseases, inflammatory conditions, and bone conditions) and/or to enhance endosomal release in a subject in need thereof is carried out by systemic administration. Suitable modes of systemic administration are disclosed supra and include, without limitation, orally, topically, transdermally, parenterally, intradermally, intrapulmonary, intramuscularly, intraperitoneally, intravenously, subcutaneously, or by intranasal instillation, by intracavitary or intravesical instillation, intraocularly, intra-arterially, intralesionally, or by application to mucous membranes.

A therapeutically effective amount of the NPC1 binding polypeptide, the NPC1 binding peptide conjugate, or NPC1 combination therapeutic for treatment of a condition as described herein (e.g., cancer, infectious diseases, neurodegenerative diseases, inflammatory conditions, and bone conditions) and/or to enhance endosomal release of a pharmaceutically active moiety in a subject as described herein, is an amount that, when administered over a particular time interval, results in achievement of one or more therapeutic benchmarks (e.g., slowing or halting of infection, inhibition of infection, cessation of symptoms, etc.). The NPC1 binding polypeptide, the NPC1 binding peptide conjugate, or NPC1 combination therapeutic comprising the same may be administered to a subject one time or multiple times. In those embodiments where the therapeutic composition is administered multiple times, it may be administered at a set interval, e.g., daily, every other day, weekly, or monthly. Alternatively, it can be administered at an irregular interval, for example on an as-needed basis based on symptoms, patient health, and the like. For example, a therapeutically effective amount may be administered once a day for one day, at least 2 days, at least 3 days, at least 4 days, at least 5 days, at least 6 days, at least 7 days, at least 10 days, or at least 15 days.

A therapeutically effective amount may vary for a subject depending on a variety of factors, including variety and extent of the symptoms, sex, age, body weight, or general health of the subject, administration mode and salt or solvate type, variation in susceptibility to the drug, the specific type of filovirus infection, and the like.

### EXAMPLES

The following examples are provided to illustrate embodiments of the present disclosure but are by no means intended to limit its scope

### Example 1—NPC1 is Upregulated in KRas Tumor Tissue

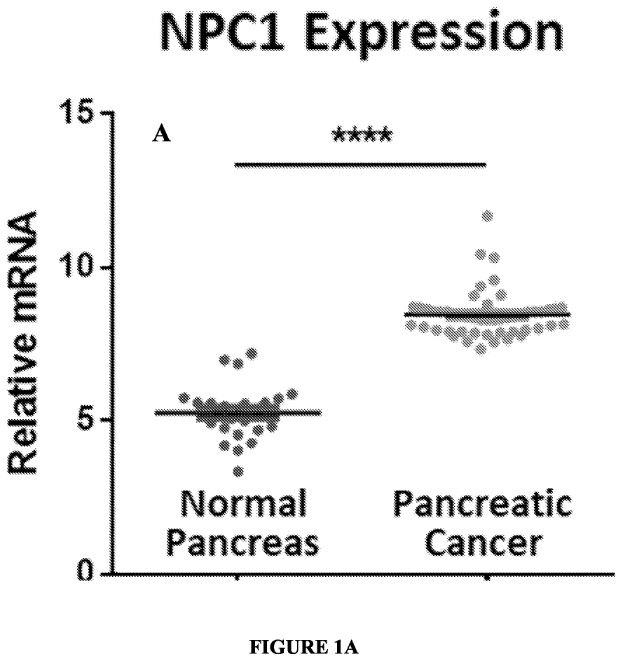

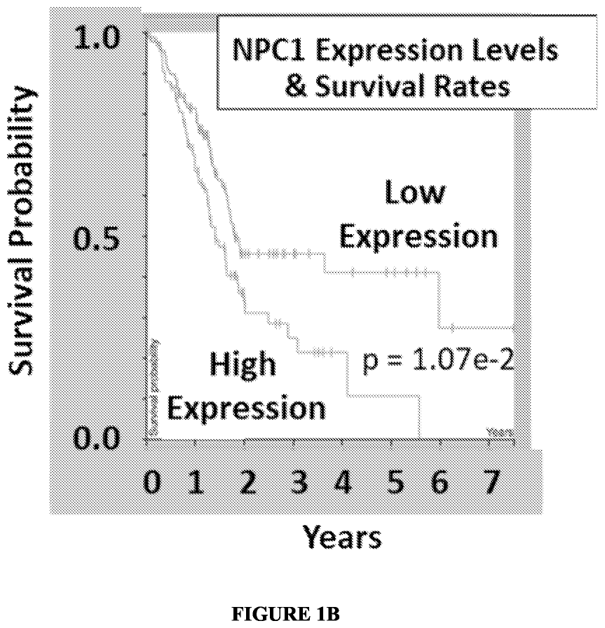

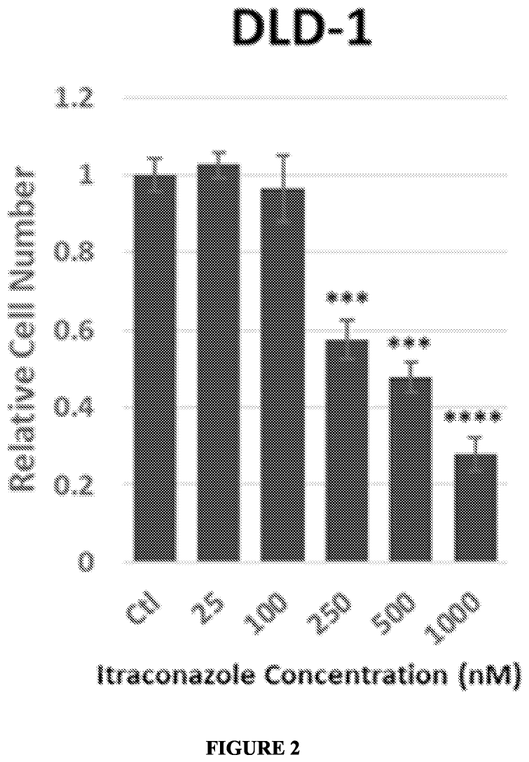

To explore a link between mutant KRas tumor tissue and NPC1 expression, pancreatic cancer tissue was analyzed as these samples are predominantly mutant KRas (˜97%). NPC1 expression was found to be elevated in tumor tissue compared to normal adjacent (FIG. 1A). Additionally, Kaplan-Meier analysis showed patients with higher NPC1 expression have a worse survival rate (FIG. 1). To test whether inhibition of NPC1 would have a growth inhibitory effect in vitro, the NPC1-targeting tool compound, itraconazole, was utilized. A dose-dependent inhibition of cell proliferation by itraconazole in mutant KRas CRC cells was observed (FIG. 2).

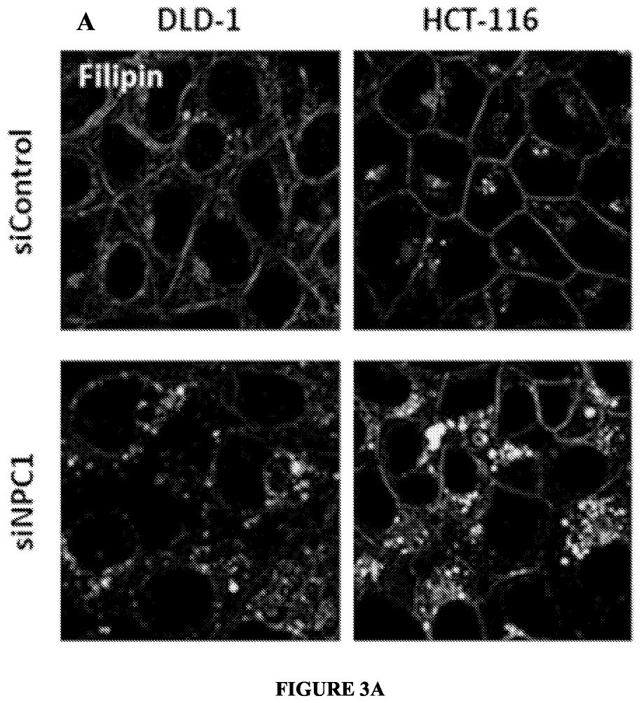

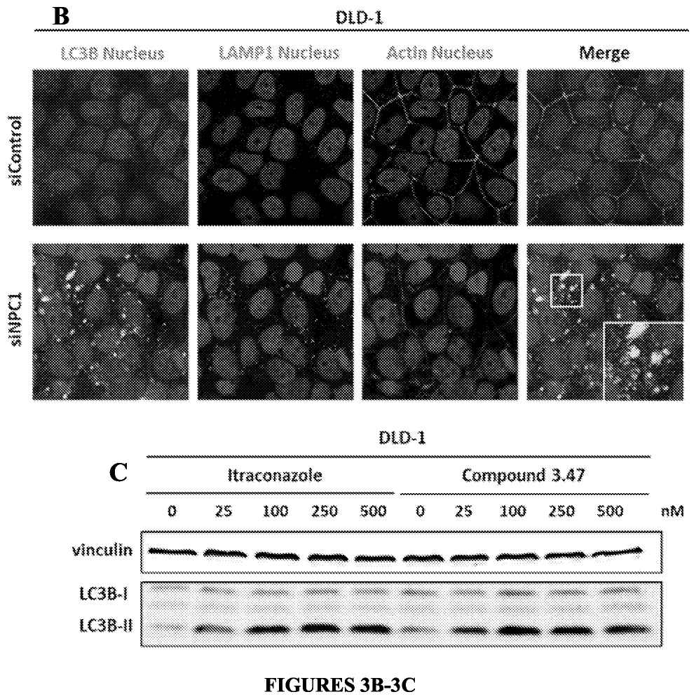

For drug discovery, a robust set of biomarkers was established for inhibition of NPC1. As shown previously in fibroblasts, siRNA-mediated knockdown of NPC1 and small molecule inhibition of NPC1 results in endosomal accumulation of cholesterol. It was verified that NPC1 has a necessary role in cholesterol trafficking in a mutant KRas cancer cell lines (FIG. 3A). Additionally, inhibition of autophagic flux has been shown to be a result of NPC1 inhibition. It was verified that autophagic flux is blocked by NPC1 knockdown and small molecule inhibition using immunofluorescence and biochemical analysis of the autophagy marker, LC3B (FIGS. 3B and 3C).

### Example 2—NPC1 Monobodies for the Treatment of Cancer

Unfortunately, the small molecule NCP1 tool compounds available are not specific to targeting cancer cells, as they freely pass through a cell's membrane. Thus, new molecular entities were developed to selectively target NPC1 in cancer cells.

Interestingly, it was discovered that monobodies, small synthetic binding proteins, are internalized by mutant Ras cancer cell lines via macropinocytosis. For this reason, NPC1 specific monobodies that are internalized by mutant-Ras expressing cancer cells were generated to facilitate the binding to and inhibition of NPC1 in the endosomal compartment.

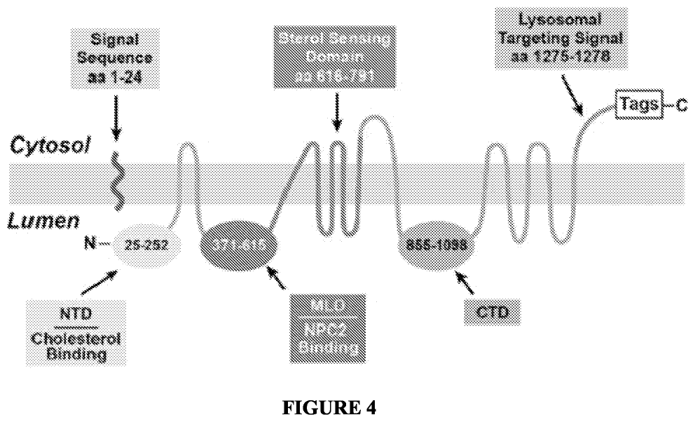

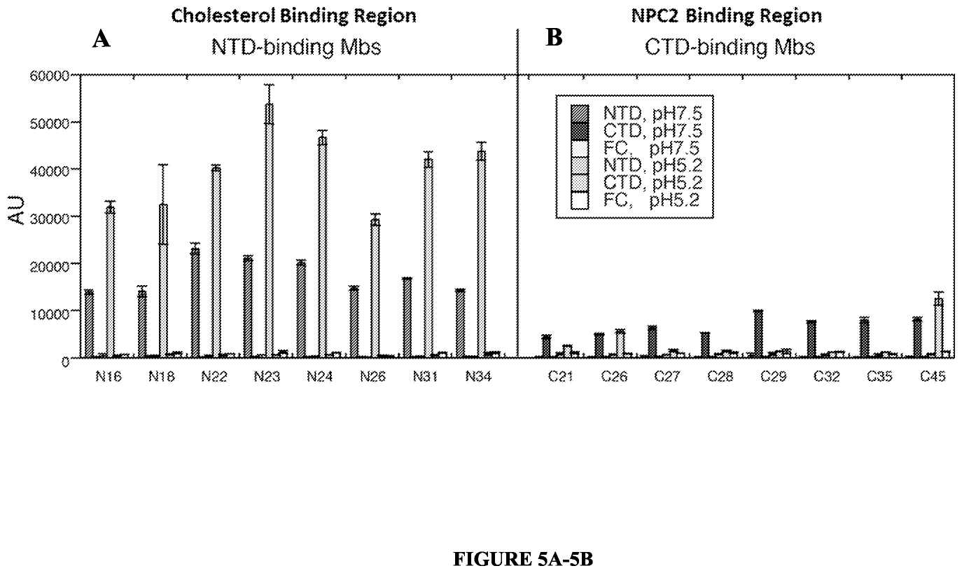

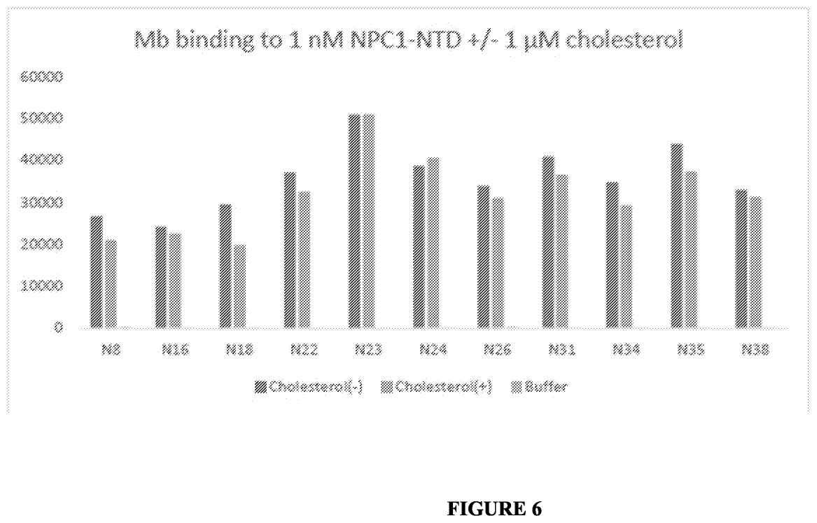

The screening of two proprietary monobody libraries produced the NPC1 monobodies described herein, i.e., binding molecules having an amino acid sequence of any one of SEQ ID NOs: 32-43) that showed strong target binding at 2.5 nM. The produced monobodies showed no difference in their ability to bind NPC1 in cholesterol-deplete or cholesterol-loaded states. Further, the NPC1 monobodies showed enhanced binding affinity to NPC1 in an acidic pH, similar to that encountered in the endosome (pH 5-6), compared to a more neutral environment (pH 7.5) (FIGS. 4-6).

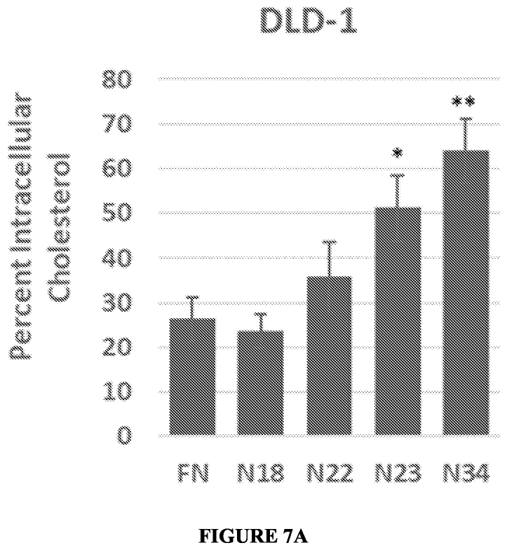

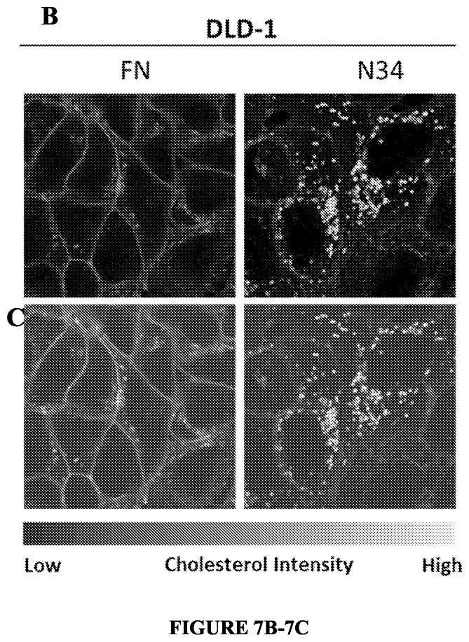

A major benefit of monobodies over antibodies is the lower cost in manufacturing. However, if monobodies need to be re-folded or aggregate during production, the cost of manufacturing can exceed that of antibodies. However, the monobodies described herein do not require re-folding and had little to no aggregation during production. Utilizing both the imaging-based and biochemical approaches, it was confirmed that the monobodies described herein inhibit NPC1 in cell culture. The NPC1-targeting monobody clones N23 (SEQ ID NO: 36) and N34 (SEQ ID NO: 40) showed the greatest inhibition of NPC1 by endosomal accumulation of cholesterol (FIG. 7). FIG. 7B DLD1 cells (Ras mutation, colon) were treated with the top monobody hits and cholesterol was measured by filipin. By targeting the cholesterol binding domain of NPC1, our preliminary data shows two monobodies (N23 and N34) caused improved cholesterol entrapment and induce vesicle disruption (FIG. 7A-7C).

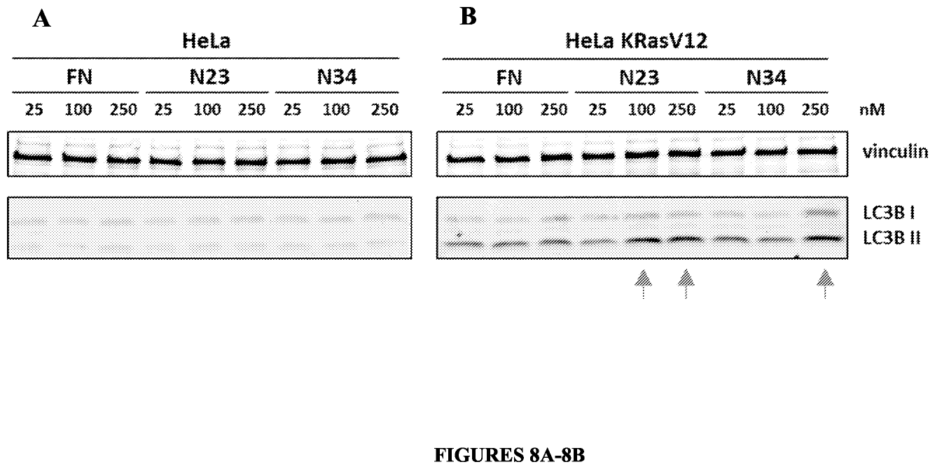

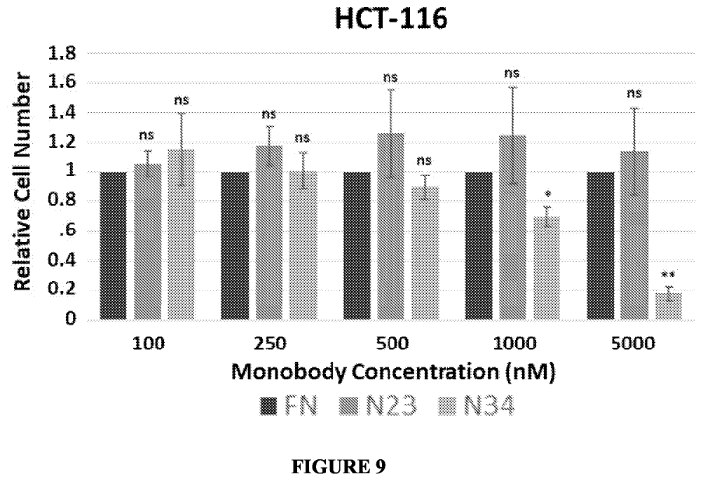

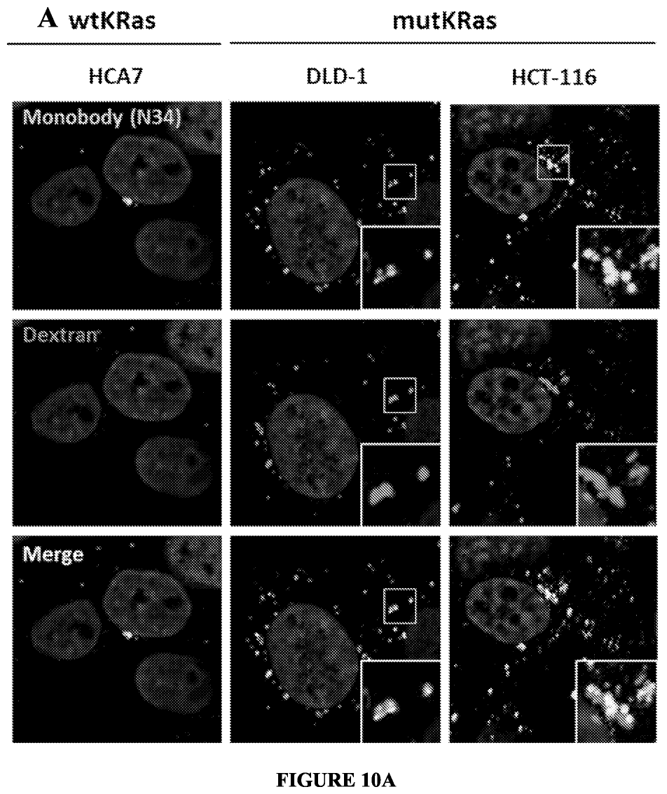

To show that the NPC1-targeting monobodies have specificity for mutant Ras cancer cells, the ability of each monobody to induce LC3B accumulation in a mutant KRas-inducible HeLa cell system was observed (FIG. 8). NPC1-targeting monobody clones N23 and N34 did not induce LC3B accumulation in HeLa cells (FIG. 8A, macropinocytosis negative) but did show an effect in HeLa KRasV12 cells (FIG. 8B, macropinocytosis positive). Comparing the candidate monobodies in CRC cell proliferation assays, N34 displayed a growth inhibitory effect while N23 had no significant inhibition (FIG. 9).

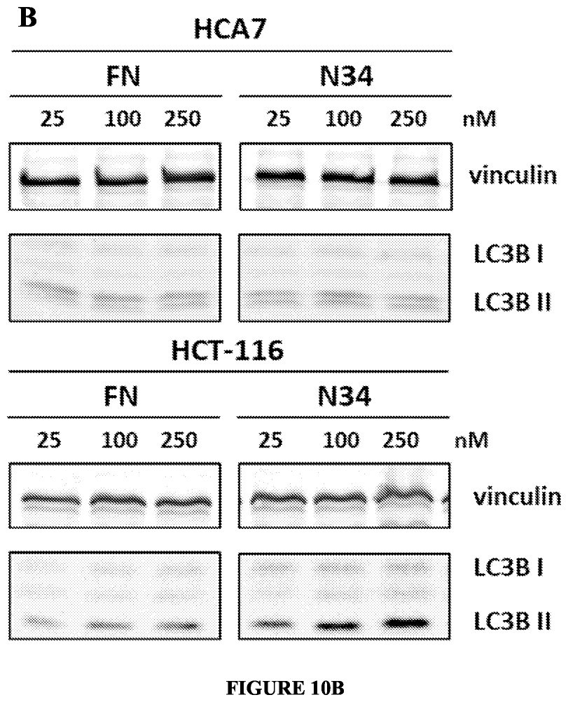

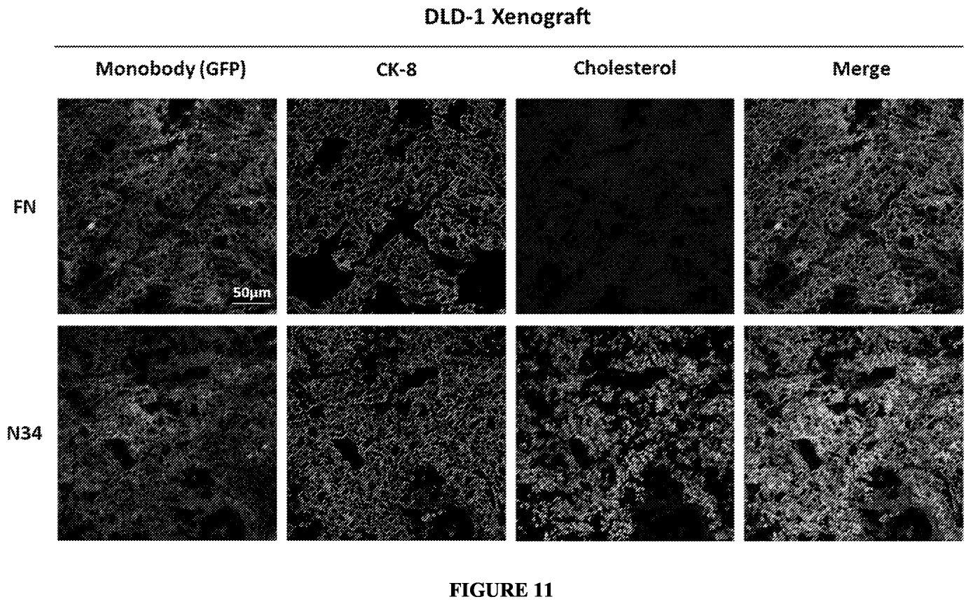

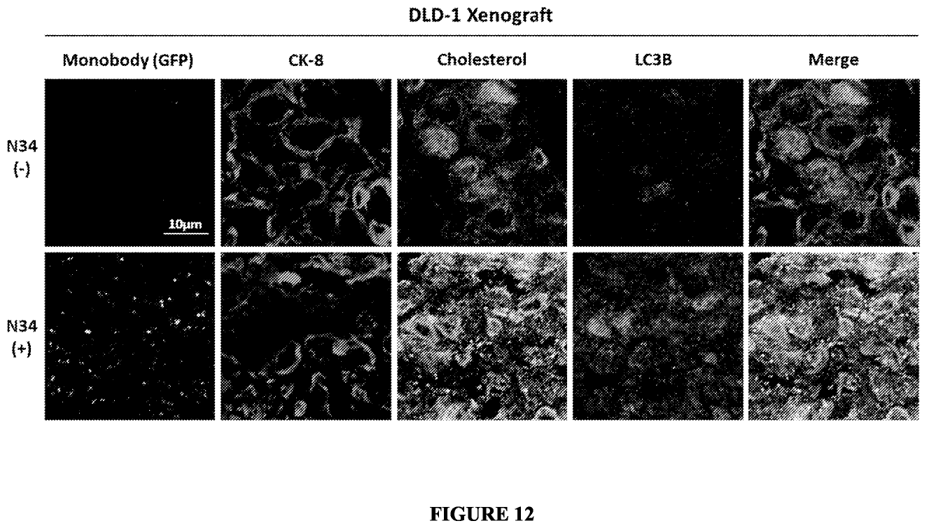

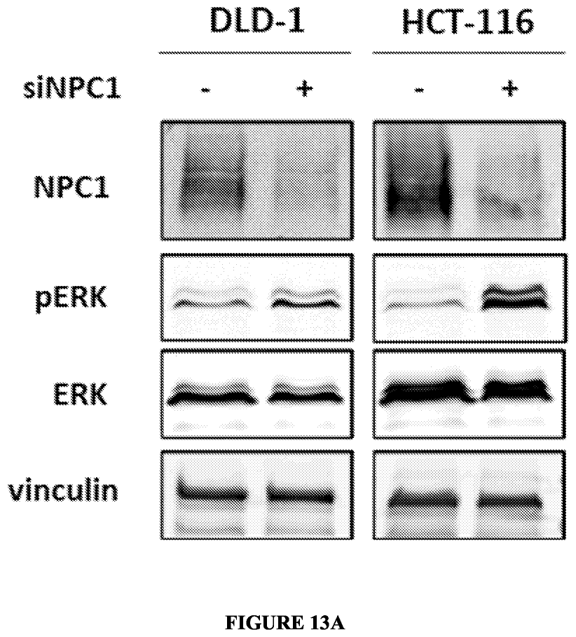

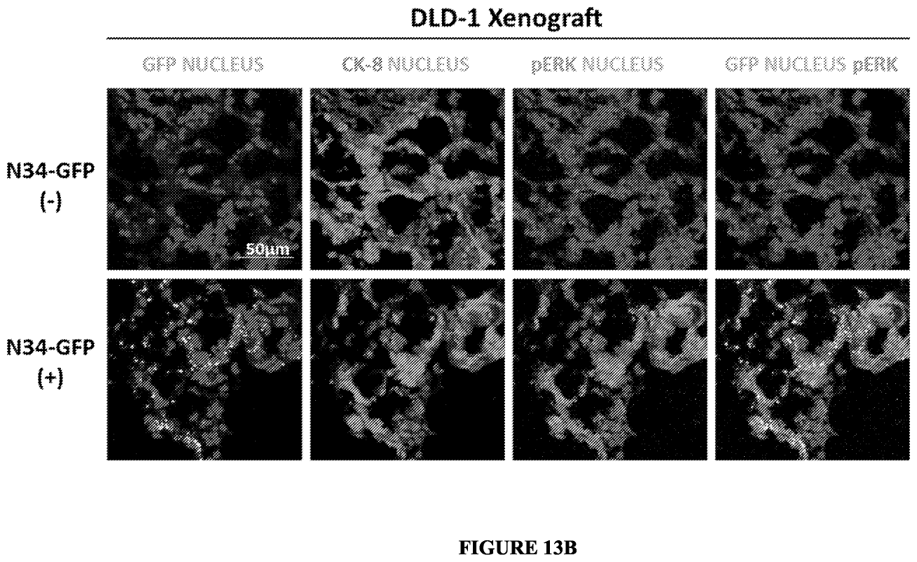

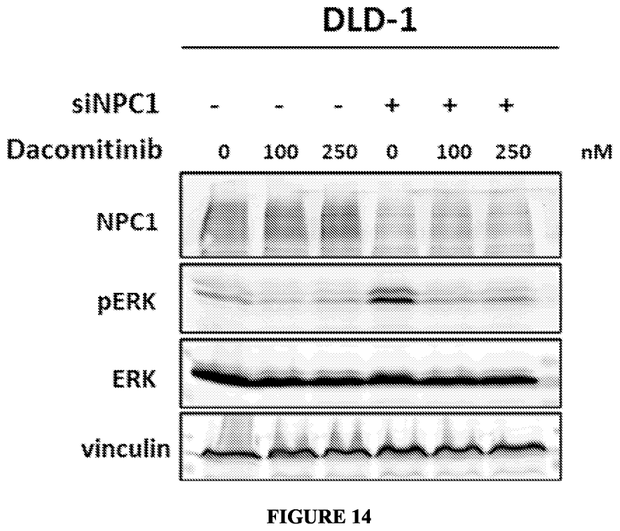

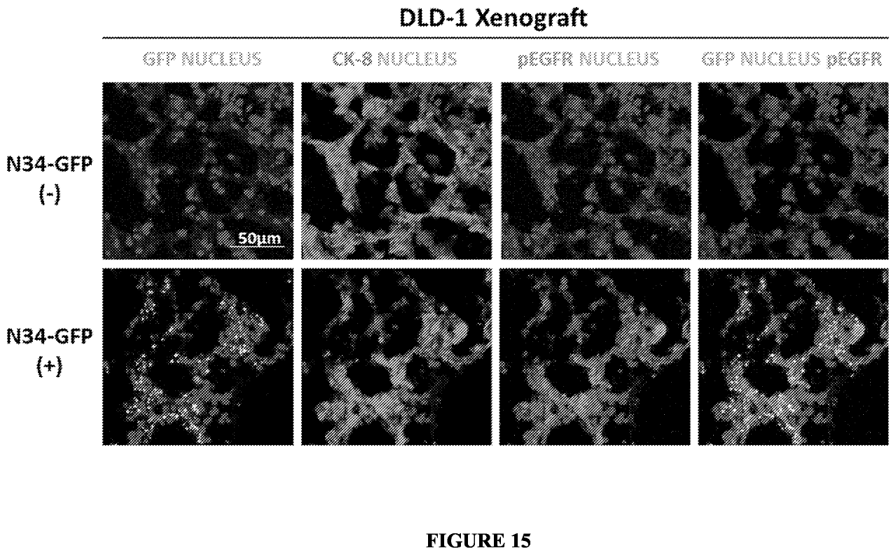

Confirmation that uptake is dependent on macropinocytosis was confirmed in CRC cell lines. Wild-type KRas CRC cells (HCA7) are macropinocytosis negative and fail to uptake the N34 monobody (FIG. 10A; left column of images). Mutant KRas CRC cells (DLD-1 and HCT-116), however, are macropinocytosis positive and efficiently internalize the N34 monobody (FIG. 10A; center and right column of images). In accordance, N34 shows a dose-dependent accumulation of LC3B in HCT-116 but not HCA7 cells (FIG. 10B), suggesting a dependence on macropinocytosis for NPC1 inhibition by NPC1-targeting monobodies. Lastly, intratumor injection of N34 monobody into xenografts, showed uptake of the monobodies in CK8-positive tumor cells (FIG. 11). Compared to non-targeting control monobody (FN), N34 positive tumor cells displayed cholesterol accumulation. Additionally, in a separate study, compared to N34-negative regions, N34-positive areas of the xenografts displayed robust cholesterol and LC3B accumulation after 2 hours post-injection (FIG. 12). It was also observed that upon NPC1 knockdown in vitro, there is ERK hyperactivation (FIG. 13A). This observation was confirmed using the N34 monobody in vivo (FIG. 13B). The hyper-activation of ERK upon NPC1 inhibition could be a result of EGFR activation, as the treatment of NPC1 knockdown cells with dacomitinib, a selective and irreversible inhibitor of EGFR, blocked ERK hyper-activation (FIG. 14). This is further supported by the observation that EGFR activation was seen upon NPC1 inhibition by N34 in vivo (FIG. 15).

### Example 3—The NPC1 Binding Peptide Conjugate Enhances Endosomal Escape of the Pharmaceutically Active Moiety

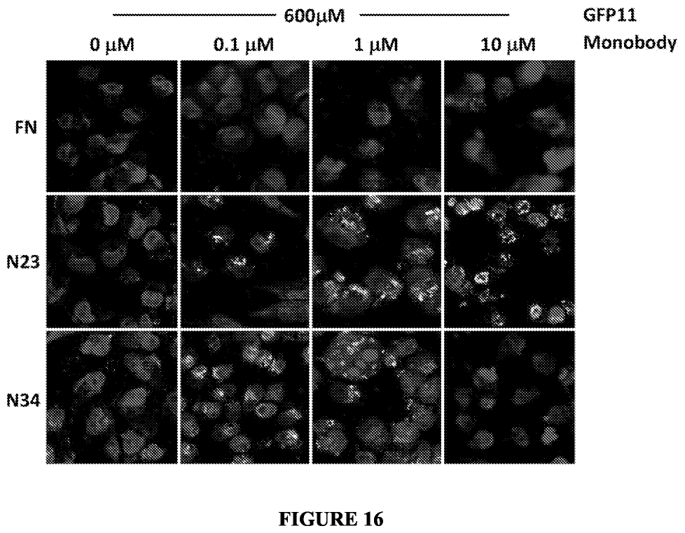

A split GFP assay was developed to measure endosomal escape of proteins. Mutant Ras PDAC MIA PaCa-2 cells were stably expressed with GFP1-10, which is missing the 11β domain needed for fluorescence, making endosomal escape of GFP110 necessary for a positive signal. NPC1 targeting and control monobodies were co-delivered with the free GFP110 domain. Fluorescence was observed with the treatment of the NPC1 targeting monobodies but not the control non-binding monobody (FN) (FIG. 16).

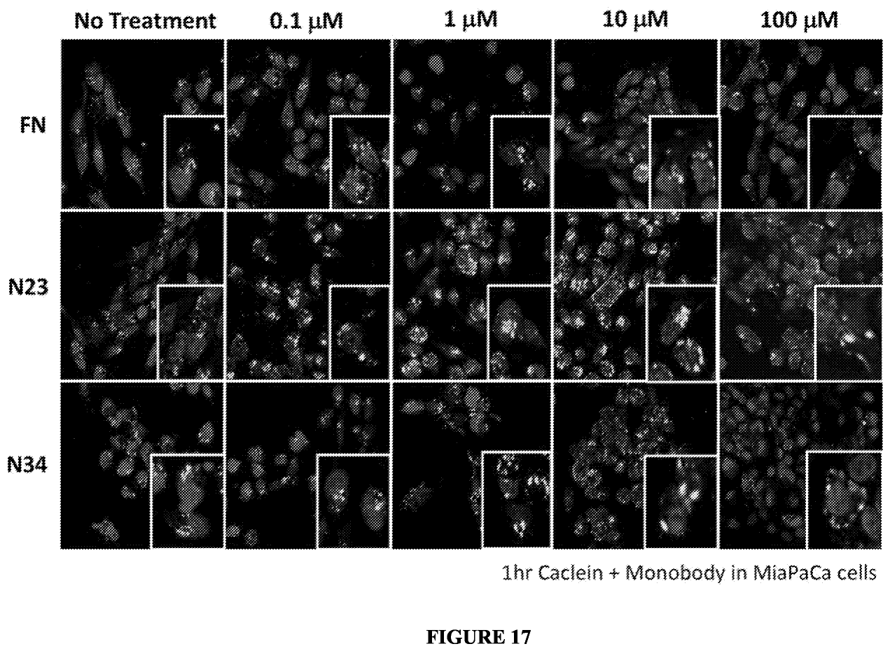

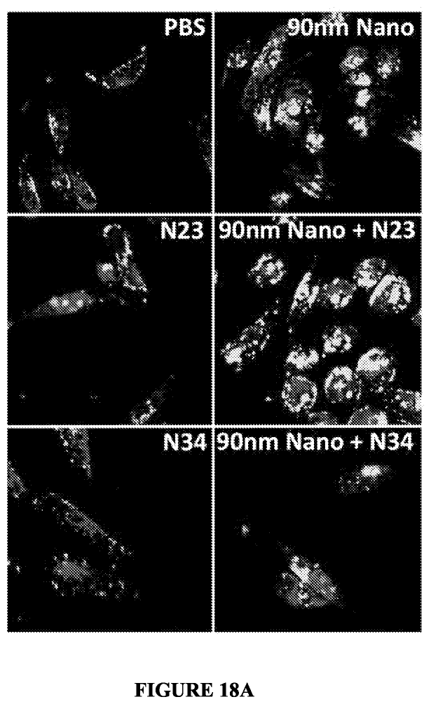

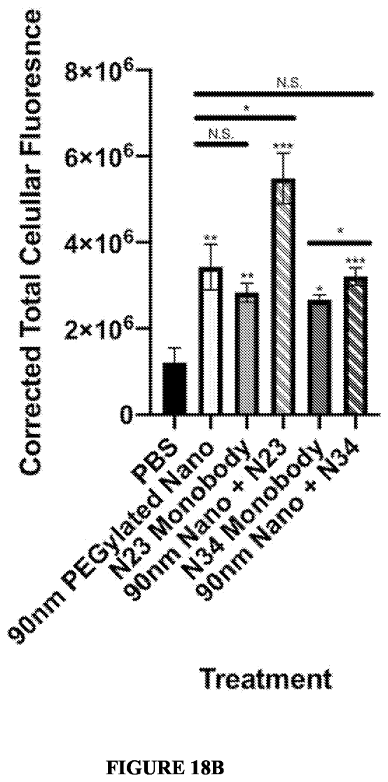

Endosome escape was again observed in a small molecule assay (FIG. 17). Calcein is a membrane impermeable, fluid phase uptake marker that is semi-quenched when in close proximity with other calcein molecules in vesicular compartments, but with intracellular release and molecule diffusion, dequenching causes an increase in cellular fluorescence. Similar to the GFP1-10 split assay, endosomal escape was seen with the co-delivery of the NPC1 targeting moieties but not with the control monobody with the increase in cellular fluorescence. The NPC1 targeting monobodies displayed increased escape when paired with nanoparticles which could be advantageous for improved nanoparticle cargo escape (FIG. 18A-18B). The NPC1 targeting monobodies have been established to induce small molecule, biologic, and nanoparticle escape.

Although preferred embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

What is claimed:

1. A Niemann-Pick disease, type C1 (NPC1) binding polypeptide, said binding polypeptide comprising a fibronectin type III (FN3) domain, said FN3 domain having a modified FG loop amino acid sequence, a modified BC loop amino acid sequence, a modified CD loop amino acid sequence, a modified DE loop amino acid sequence, or a combination thereof, wherein said one or more modified loop sequences enable binding to NPC1.

2. The binding polypeptide of claim 1, wherein the modified FG loop amino acid sequence is selected from any one of SEQ ID NOs: 2-13.

3. The binding polypeptide of claim 1 and claim 2, wherein the modified BC loop amino acid sequence is selected from any one of SEQ ID NOs: 15-21.

4. The binding polypeptide of any one of claims 1-3, wherein the modified CD loop amino acid sequence is selected from any one of SEQ ID NOs: 23-28.

5. The binding polypeptide of any one of claims 1-4, wherein the modified DE loop amino acid sequence is selected from any one of SEQ ID NOs: 30-32.

6. The binding polypeptide of any one of claims 1-5, wherein the FN3 domain is a human fibronectin type III tenth domain (¹⁰Fn3) of SEQ ID NO:1 comprising the at least one modified loop amino acid sequences.

7. The binding polypeptide of claim 6, wherein the ¹⁰Fn3 domain further comprises an amino acid substitution in one or more of the C, D, E, or F beta-strands.

8. The binding polypeptide of claim 7, wherein the amino acid substitution is at one or more residues selected from R33, E47, T49, and A74 of SEQ ID NO: 1.

9. The binding polypeptide of claim 8, wherein the amino acid substitution at R33 is selected from the group consisting of R33V, R33D, and R33F.

10. The binding polypeptide of claim 8, wherein the amino acid substitution at E47 is selected from the group consisting of E47T and E47K.

11. The binding polypeptide of claim 8, wherein the amino acid substitution at T49 is selected from the group consisting of T49K and T49A.

12. The binding polypeptide of claim 8, wherein the amino acid substitution at A74 is A74T.

13. The binding polypeptide of claim 6, further comprising an amino acid substitution at one or more resides selected from D3, R6 and D7 of SEQ ID NO: 1.

14. The binding polypeptide of any one of claims 1-13, wherein the FN3 domain comprises:
(i) a modified FG loop amino acid sequence of SEQ ID NO: 2, a modified BC loop amino acid sequence of SEQ ID NO: 15, and a modified DE loop amino acid sequence of SEQ ID NO: 30 (N8);
(ii) a modified FG loop amino acid sequence of SEQ ID NO: 3, a modified BC loop amino acid sequence of SEQ ID NO: 16, and a modified DE loop amino acid sequence of SEQ ID NO: 30 (N16);
(iii) a modified FG loop amino acid sequence of SEQ ID NO: 4, a modified BC loop amino acid sequence of SEQ ID NO: 17, and a modified DE loop amino acid sequence of SEQ ID NO: 30 (N18);
(iv) a modified FG loop amino acid sequence of SEQ ID NO: 5, a modified BC loop amino acid sequence of SEQ ID NO: 18, and a modified CD loop amino acid sequence of SEQ ID NO: 23 (N22);
(v) a modified FG loop amino acid sequence of SEQ ID NO: 6, a modified BC loop amino acid sequence of SEQ ID NO: 19, and a modified CD loop amino acid sequence of SEQ ID NO: 23 (N23);
(vi) a modified FG loop amino acid sequence of SEQ ID NO: 7, a modified BC loop amino acid sequence of SEQ ID NO: 18, and a modified CD loop amino acid sequence of SEQ ID NO: 24 (N24);
(vii) a modified FG loop amino acid sequence of SEQ ID NO: 8, a modified BC loop amino acid sequence of SEQ ID NO: 18, and a modified CD loop amino acid sequence of SEQ ID NO: 25 (N26);
(viii) a modified FG loop amino acid sequence of SEQ ID NO: 9, a modified BC loop amino acid sequence of SEQ ID NO: 18, and a modified CD loop amino acid sequence of SEQ ID NO: 26 (N31);
(ix) a modified FG loop amino acid sequence of SEQ ID NO: 10, a modified BC loop amino acid sequence of SEQ ID NO: 18, and a modified CD loop amino acid sequence of SEQ ID NO: 26 (N34)
(x) a modified FG loop amino acid sequence of SEQ ID NO: 11, a modified BC loop amino acid sequence of SEQ ID NO: 20, and a modified CD loop amino acid sequence of SEQ ID NO: 24 (N35);
(xi) a modified FG loop amino acid sequence of SEQ ID NO: 12, a modified BC loop amino acid sequence of SEQ ID NO: 21, and a modified CD loop amino acid sequence of SEQ ID NO: 27 (N38); and
(xii) a modified FG loop amino acid sequence of SEQ ID NO: 13, a modified BC loop amino acid sequence of SEQ ID NO: 20, and a modified CD loop amino acid sequence of SEQ ID NO: 28 (C45).

15. The binding polypeptide of any one of claims 1-14, wherein the FN3 domain comprises an amino acid sequence that is at least 80% identical to an amino acid sequence selected from the group consisting of SEQ ID NOs: 32-43.

16. The binding polypeptide of any one of claims 1-14, wherein the FN3 domain comprises an amino acid sequence that is at least 90% identical to an amino acid sequence selected from the group consisting of SEQ ID NOs: 32-43.

17. The binding polypeptide of any one of claims 1-14, wherein the FN3 domain comprises an amino acid sequence that is at least 95% identical to an amino acid sequence selected from the group consisting of SEQ ID NOs: 32-43.

18. The binding polypeptide of any one of claims 1-14, wherein the FN3 domain comprises an amino acid sequence selected from the group consisting of SEQ ID NOs:
32-43.

19. A Niemann-Pick disease, type C1 (NPC1) binding peptide conjugate, said conjugate comprising:
a first portion, said first portion comprising the binding polypeptide of any one of claims 1-18 and
a second portion coupled to said first portion, said second portion selected from a pharmaceutically active moiety, a diagnostic moiety, a half-life extending moiety, a delivery vehicle, a prodrug, a second binding molecule, a polymer, and a non-binding protein.

20. The NPC1 binding peptide conjugate of claim 19, wherein the second portion is a pharmaceutically active moiety.

21. The NPC1 binding peptide conjugate of claim 20, wherein the pharmaceutically active moiety is selected from the group consisting of a small molecule, a nucleic acid molecule, an antibody or antigen binding fragment thereof, an antibody derivative, a protein or polypeptide fragment thereof, and a proteolysis targeting chimera (PROTAC).

22. The NPC1 binding peptide conjugate of claim 20 or claim 21, wherein the pharmaceutically active moiety is a cancer therapeutic.

23. The NPC1 binding peptide conjugate of claim 22, wherein the cancer therapeutic is selected from an antimetabolite, an alkaloid, an alkylating agent, an anti-mitotic agent, an antitumor antibiotic, a DNA binding drug, a toxin, an anti-proliferative drug, a DNA antagonist, a radionuclide, a thermoablative agent, a proteolysis targeting chimera (PROTAC), and a nucleic acid inhibitor.

24. The NPC1 binding peptide conjugate of claim 23, wherein the alkaloid is selected from the group consisting of duocarmycin, docetaxel, etoposide, irinotecan, paclitaxel, teniposide, topotecan, vinblastine, vincristine, vindesine, and analogs and derivatives thereof.

25. The NPC1 binding peptide conjugate of claim 23, wherein the alkylating agent is selected from the group consisting of busulfan, improsulfan, piposulfan, benzodepa, carboquone, meturedepa, uredepa, altretamine, triethylenemelamine, triethylenephosphoramide, triethylenethiophosphorarnide, chlorambucil, chloranaphazine, cyclophosphamide, estramustine, ifosfamide, mechlorethamine, mechlorethamine oxide HCl, melphalan, novemebichin, perfosfamide phenesterine, prednimustine, trofosfamide, uracil mustard, carmustine, chlorozotocin, fotemustine, lomustine, nimustine, semustine ranimustine, dacarbazine, mannomustine, mitobronitol, mitolactol, pipobroman, temozolomide, and analogs and derivatives thereof.

26. The NPC1 binding peptide conjugate of claim 23, wherein the antitumor antibiotic is selected from the group consisting of aclacinomycin, actinomycin, anthramycin, azaserine, bleomycin, cactinomycin, calicheamicin, carubicin, carzinophilin, cromomycin, dactinomycin, daunorubicin, 6-diazo-5-oxo-1-norleucine, doxorubicin, epirabicin, idarubicin, menogaril, mitomycin, mycophenolic acid, nogalamycine, olivomycin, peplomycin, pirarubicin, plicamycin, porfiromycin, puromycine, pyrrolobenzodiazepine, streptonigrin, streptozocin, tubercidin, zinostatin, zorubicin, and analogs and derivatives thereof.

27. The NPC1 binding peptide conjugate of claim 23, wherein the antimetabolite is selected from the group consisting of from SN-38, denopterin, edatrexate, mercaptopurine (6-MP), methotrexate, piritrexim, pteropterin, pentostatin (2′-DCF), tomudex, trimetrexate, cladridine, fludarabine, thiamiprine, ancitabine, azacitidine, 6-azauridine, carmofur, cytarabine, doxifluridine, emitefur, floxuridine, fluorouracil, gemcitabine, tegafur, hydroxyurea, urethane, and analogs and derivatives thereof.

28. The NPC1 binding peptide conjugate of claim 23, wherein the anti-proliferative drug is selected from the group consisting of aceglatone, amsacrine, bisantrene, camptothecin, defosfamide, demecolcine, diaziquone, diflomotecan, eflornithine, elliptinium acetate, etoglucid, etopside, fenretinide, gallium nitrate, hydroxyurea, lamellarin D, lonidamine, miltefosine, mitoguazone, mitoxantrone, mopidamol, nitracrine, pentostatin, phenamet, podophillinic acid 2-ethyl-hydrazide, procarbazine, razoxane, sobuzoxane, spirogermanium, teniposide, tenuazonic acid, triaziquone 2,2′,2″-trichlorotriethylamine, and analogs and derivatives thereof.

29. The NPC1 binding peptide conjugate of claim 23, wherein the antimitotic agent is selected from the group consisting of an auristatin, a maytansinoid, a dolastatin, a tubulysin, a taxane, an epothilone, a vinca alkaloid, and analogs and derivatives thereof.

30. The NPC1 binding peptide conjugate of claim 20 or claim 21, wherein the pharmaceutically active moiety is an immunomodulatory agent.

31. The NPC1 binding peptide conjugate of claim 30, wherein the immunomodulatory agent is a macrophage type-1 stimulating agent.

32. The NPC1 binding peptide conjugate of claim 31, wherein the macrophage type-1 stimulating agent is selected from the group consisting of paclitaxel, a colony stimulating factor −1 (CSF-1) receptor antagonist, an IL-10 receptor antagonist, a Toll-like receptor (TLR)-2 agonist, a TLR-3 agonist, a TLR-4 agonist, a TLR-7 agonist, a TLR-8 agonist, and a TLR-9 agonist.

33. The NPC1 binding peptide conjugate of claim 30, wherein the immunomodulatory agent is a macrophage type-2 stimulating agent.

34. The NPC1 binding peptide conjugate of claim 33, wherein the macrophage type-2 stimulating agent is selected from the group consisting of IL-33, IL-4 receptor agonists, glucocorticoids, IL-10 receptor agonist, and IL-1 receptor agonist.

35. The NPC1 binding peptide conjugate of claim 30, wherein the immunomodulatory agent is an T cell stimulating agent.

36. The NPC1 binding peptide conjugate of claim 35, wherein the T cell stimulating agent is a stimulator of interferon genes (STING) agonist.

37. The NPC1 binding peptide conjugate of claim 30, wherein the immunomodulatory agent is a dendritic cell stimulating agent.

38. The NPC1 binding peptide conjugate of claim 37, wherein the dendritic cell stimulating agent is selected from the group consisting of CpG oligonucleotide, imiquimod, camptothecin, colchicine, podophyllotoxin, and derivatives thereof.

39. The NPC1 binding peptide conjugate of claim 30, wherein the immunomodulatory agent is a neutrophil stimulating agent.

40. The NPC1 binding peptide conjugate of claim 39, wherein the neutrophil stimulating agent is a recombinant granulocyte colony stimulating factor protein (filgrastim) or a pegylated recombinant granulocyte colony stimulating factor protein.

41. The NPC1 binding peptide conjugate of claim 21, wherein the pharmaceutically active moiety is a nucleic acid molecule.

42. The NPC1 binding peptide conjugate of claim 41, wherein the nucleic acid molecule is selected from the group consisting of an siRNA, an aptamer, an miRNA, an immunostimulatory oligonucleotide, a splice-switching oligonucleotide, and guide RNA.

43. The NPC1 binding peptide conjugate of any one of claims 20-42, wherein the pharmaceutically active moiety is coupled to a delivery vehicle.

44. The NPC1 binding peptide conjugate of claim 19, wherein the second portion of the conjugate is a delivery vehicle.

45. The NPC1 binding peptide conjugate of claim 43 or 44, wherein the delivery vehicle is selected from a nanoparticle, a polymer-based particle, and a lipid-based particle.

46. The NPC1 binding peptide conjugate of claim 19, wherein the second portion is a diagnostic moiety.

47. The NPC1 binding peptide conjugate of claim 46, wherein the diagnostic moiety is selected from the group consisting of a fluorescent dye, a radioisotope, a contrast agent suitable for imaging, a radionucleotide with chelator, and a photosensitizer.

48. An isolated polynucleotide encoding the NPC1 binding polypeptide of any one of claims 1-18 or the NPC1 binding peptide conjugate of claim 19.

49. A vector comprising the isolated polynucleotide of claim 48.

50. A host cell comprising the vector of claim 49.

51. A pharmaceutical composition comprising:
the binding polypeptide of any one of claims 1-18, the NPC1 binding peptide conjugate of any one of claims 19-47, the isolated polynucleotide of claim 48, or the vector of claim 49 and
a pharmaceutical carrier.

52. A combination therapeutic comprising:
a binding polypeptide of an any one of claims 1-18 and
a pharmaceutically active moiety.

53. The combination therapeutic of claim 52, wherein the pharmaceutically active moiety is selected from the group consisting of a small molecule, a nucleic acid molecule, an antibody or antigen binding fragment thereof, an antibody derivative, a protein or polypeptide fragment thereof, and a proteolysis targeting chimera (PROTAC).

54. The combination therapeutic of claim 52 or claim 53, wherein the pharmaceutically active moiety is a cancer therapeutic.

55. The combination therapeutic of claim 54, wherein the cancer therapeutic is a chemotherapeutic.

56. The combination therapeutic of claim 55, where the chemotherapeutic is selected from cyclophosphamide, gemcitabine, vorinostat, temozolomide, bortezomib, carmustine, and paclitaxel.

57. The combination therapeutic of claim 54, wherein the cancer therapeutic is an immune checkpoint inhibitor.

58. The combination therapeutic of claim 57, wherein the immune checkpoint inhibitor is selected from a CTLA-4 inhibitor, a PD-1 inhibitor, and a PD-L1 inhibitor.

59. The combination therapeutic of claim 54, wherein the cancer therapeutic is selected from an epidermal growth factor (EGFR) inhibitor and an mTOR inhibitor.

60. A method for treating cancer in a subject, said method comprising:
administering, to the subject having cancer, the pharmaceutical composition of claim 51 in an amount effective to treat the cancer.

61. The method of claim 60, wherein the cancer is characterized by cancerous cells having enhanced macropinocytosis relative to their corresponding non-cancerous cells.

62. The method of claim 60, wherein the cancer is characterized by cancerous cells having an oncogenic mutation in H-ras, N-ras, or K-ras.

63. The method of any one of claims 60-62, wherein the cancer is pancreatic cancer, lung cancer, breast cancer, colon cancer, glioma, solid tumor, melanoma, glioblastoma multiforme, leukemia, renal cell carcinoma, hepatocellular carcinoma, prostate cancer, and myeloma.

64. The method of claim 60, wherein said method further comprising:
administering a cancer therapeutic in conjunction with said pharmaceutical composition.

65. The method of claim 64, wherein the cancer therapeutic is a chemotherapeutic.

66. The method of claim 65, wherein the chemotherapeutic is selected from cyclophosphamide, gemcitabine, vorinostat, temozolomide, bortezomib, carmustine, paclitaxel, mitoxantrone, and capecitabine.

67. The method of claim 64, wherein the cancer therapeutic is an immune checkpoint inhibitor.

68. The method of claim 67, wherein the immune checkpoint inhibitor is selected from a CTLA-4 inhibitor, a PD-1 inhibitor, and a PD-L1 inhibitor.

69. The method of claim 64, wherein the cancer therapeutic is selected from an epidermal growth factor (EGFR) inhibitor and an mTOR inhibitor.

70. The method of claim 60, wherein said method further comprising:
administering said pharmaceutical composition in conjunction with radiation therapy.

71. A method for treating an infectious disease in a subject, said method comprising:
administering, to the subject having an infectious disease, the binding polypeptide of any one of claims 1-18 or the NPC1 binding peptide conjugate of claim 19 in an amount effective to treat the infectious disease.

72. The method of claim 71, wherein the infectious disease is caused by a filovirus.

73. The method of claim 72, wherein the filovirus is ebola virus or Marburg virus

74. The method of claim 71, wherein the infectious disease is caused by a coronavirus.

75. The method of claim 74, wherein the coronavirus is Severe Acute Respiratory Syndrome Coronavirus-2 (SARS-CoV-2) or Middle East Respiratory Syndrome Coronavirus (MERS-CoV).

76. A method of enhancing endosomal release of a pharmaceutically active moiety in a subject in need thereof, said method comprising:
administering, to said subject, a NPC1 binding peptide conjugate, wherein said peptide conjugate comprises:
a first portion, said first portion comprising the binding polypeptide of any one of claims 1-18 and
a second portion, coupled to said first portion, said second portion comprising the pharmaceutically active moiety.

77. A method of enhancing endosomal release of a pharmaceutically active moiety in a subject in need thereof, said method comprising:
administering, to said subject, a combination therapeutic, said combination therapeutic comprising:
the NPC1 binding polypeptide of an any one of claims 1-18 and
the pharmaceutically active moiety.

78. The method of claim 76 or claim 77, wherein the pharmaceutically active moiety is selected from the group consisting of a small molecule, a nucleic acid molecule, an antibody or antigen binding fragment thereof, an antibody derivative, a protein or polypeptide fragment thereof, and a proteolysis targeting chimera (PROTAC).

79. The method of any one of claims 76-78, wherein the subject has a neurodegenerative disease and the pharmaceutically active moiety is suitable for treating said neurodegenerative disease.

80. The method of claim 79, wherein the neurodegenerative disease is selected from the group consisting of amyotrophic lateral sclerosis, Parkinson's disease, Huntington's disease, and Alzheimer's disease.

81. The method of any one of claims 76-78, wherein the subject has an inflammatory condition and the pharmaceutically active moiety is suitable for treating said inflammatory condition.

82. The method of claim 81, wherein the inflammatory condition is rheumatoid arthritis or atherosclerosis.

83. The method of any one of claims 76-78, wherein the subject has a bone condition and the pharmaceutically active moiety is suitable for treating said bone condition.

84. The method of claim 83, wherein the bone condition is osteoporosis or Paget's Bone disease.

85. The method of any one of claims 76-78, wherein the subject has cancer and the pharmaceutically active moiety is suitable for treating said cancer.

86. The method of claim 85, wherein the cancer is associated with RAS-pathway activation.
